(19) United States
(12) Patent Application Publication
(10) Pub. No.: US 2026/0224630 A1
(43) Pub. Date: Aug. 6, 2026

(54) DEVICES AND METHODS FOR PREPARING PLATELET RICH FIBRIN MATRIX AND USES THEREOF

(71) Applicant: Tiger Wound Care Medical, LLC, Conshohocken, PA (US)

(72) Inventors: Lucas FORNACE, Plano, TX (US); Jeff GREINER, Plano, TX (US)

(21) Appl. No.: 19/532,827

(22) Filed: Feb. 6, 2026

Related U.S. Application Data
(60) Provisional application No. 63/754,984, filed on Feb. 6, 2025.

(51) Int. Cl. A61K 35/19; A61K 38/36; A61K 47/02; A61K 47/12; A61K 47/54

(52) U.S. Cl. CPC A61K 35/19; A61K 38/36; A61K 47/02; A61K 47/12; A61K 47/545; A61K 47/549

(57) ABSTRACT

Devices, kits, and methods provide enhanced blood separation to yield a composition of platelet rich fibrin matrix (PRFM). The devices may be pre-filled with layered components that facilitate the preparation of activation (e.g., full or partial activation) of platelet rich plasma (PRP) to produce a PRFM. The devices, kits, and methods reduce the number of steps involved with preparation of PRFM, reducing the need for external processing and mixing steps and reagents while providing a reproduceable methodology.

### PRIORITY

The present application claims priority to U.S. Provisional Patent App. No. 63/754,984, filed on Feb. 6, 2025, the entire disclosure of which is incorporated herein by reference.

### BACKGROUND

Wound healing is a significant focus of the medical field, as treatments are being developed to address various types of wounds, such as surgical wounds, lacerations, abrasions, burns, and traumatic injuries. Various materials, chemical and biological, are used for this purpose and several systems have been developed to ensure effective delivery of wound healing agents to the different layers of the skin and tissue.

Platelet rich fibrin matrix (PRFM) has been used for cosmetic purposes and for the treatment of injuries and conditions, in particular those relating to the healing and regeneration of skin, bone, and other tissue. PRFM is a fibrin matrix that contains a concentrated mixture of platelets and growth factors that play a role in the wound healing process.

There exists a need for improved devices, systems, and methods for the preparation and collection of PRFM.

### SUMMARY OF THE DISCLOSURE

The present disclosure features devices, methods, and kits for preparing PRFM from a blood sample.

A first aspect of the present disclosure is directed to a method comprising: introducing a blood sample into a tube, wherein the blood sample, when in the tube, is separated from a stabilizing agent and a coagulating agent with at least one gel layer; centrifuging the tube to pass red blood cells of the blood sample through the at least one gel layer and to form a mixture of the stabilizing agent, the coagulating agent, and plasma of the blood sample positioned above the at least one gel layer; and resuspending platelets of the blood sample into the mixture to form a platelet rich fibrin matrix (PRFM) layer in the tube.

In some embodiments, the method may include one or more of the following features.

The method may include removing at least a portion of the PRFM layer from the tube in a fluid state with a syringe; and applying the at least the portion of the PRFM layer to a wound site. The PRFM layer may be removed from the tube having a viscosity less than about 100 cP. The method may include removing at least a portion of the PRFM layer from the tube in a gel state; and applying the at least the portion of the PRFM layer to a wound site. The PRMF layer may be removed from the tube having a viscosity greater than about 100 cP. The coagulating agent may activate the platelets to form the PRFM layer, and the stabilizing agent stabilizes the PRFM layer. The at least one gel layer may include a first gel layer separating the coagulating agent from the stabilizing agent and a second gel layer separating the coagulating agent and the stabilizing agent from the blood sample. The method may include mixing the blood sample with an anticoagulant in the tube before centrifuging. The anticoagulant may include anticoagulant citrate dextrose A (ACD-A), buffered citrate, sodium citrate, citrate phosphate dextrose (CPD), ethylenediaminetetraacetic acid (EDTA), heparin, oxolate, a derivative thereof, or any combination thereof. The anticoagulant may be ACD-A. The stabilizing agent may be ascorbic acid. The coagulating agent may be a calcium salt. The calcium salt may be calcium gluconate and/or calcium chloride. The at least one gel layer may be at least one thixotropic gel. The tube may be prefilled with the stabilizing agent, the coagulating agent, and the at least one gel layer.

The tube may be prefilled with an anti-coagulant agent. The tube may contain a vacuum and is closed by a sealing cap. The stabilizing agent may be prefilled in the tube at a concentration of from about 400 mg/mL to about 600 mg/mL, inclusive. The coagulating agent may be prefilled in the tube at a concentration of from about 50 mg/mL to about 150 mg/mL, inclusive. The centrifuging may be at an acceleration of from about 1000 g to about 4500 g, inclusive. The centrifuging may be for a time of from about 3 minutes to about 12 minutes, inclusive. The centrifuging may be the only centrifugation.

A second aspect of the present disclosure is directed to a pre-filled tube configured to prepare a platelet rich fibrin matrix (PRFM) layer, wherein the pre-filled tube comprises: a first drug layer comprising a coagulating agent; a second drug layer comprising a stabilizing agent; and a gel layer separating the first drug layer and the second drug layer.

In some embodiments, the pre-filled tube of the second aspect may include one or more of the following features. The pre-filled tube may comprise: a third drug layer comprising an anticoagulant; and a second gel layer separating the third drug layer from the first drug layer and the second drug layer. The anticoagulant may comprise anticoagulant citrate dextrose A (ACD-A), buffered citrate, sodium citrate, citrate phosphate dextrose (CPD), ethylenediaminetetraacetic acid (EDTA), heparin, oxolate, a derivative thereof, or any combination thereof. The anticoagulant may be ACD-A. The coagulating agent may comprise a calcium salt. The calcium salt may be calcium gluconate and/or calcium chloride. The coagulating agent may have a concentration of from about 50 mg/mL to about 150 mg/mL, inclusive. The stabilizing agent may be ascorbic acid. The stabilizing agent may have a concentration of from about 400 mg/mL to about 600 mg/mL, inclusive. The first drug layer may be closer to a distal end of the pre-filled tube than the second drug layer. The second drug layer may be closer to a distal end of the pre-filled tube than the first drug layer. The pre-filled tube may be sealed at a proximal end with a sealing cap to create a vacuum inside of the pre-filled tube. The pre-filled tube may contain an air pressure from about negative 50 kPa to about negative 101 kPa, inclusive.

A third aspect of the present disclosure is directed to a pre-filled tube configured to prepare a platelet rich fibrin matrix (PRFM) layer, wherein the pre-filled tube contains five layers of liquid, gel, or solid material and no more than five layers of liquid, gel, or solid material, the five layers of liquid, gel, or solid material comprising: a first drug layer comprising a coagulating agent; a second drug layer comprising a stabilizing agent; a third drug layer comprising an anticoagulant; a first gel layer separating the first drug layer and the second drug layer; and a second gel layer separating the third drug layer from the first drug layer and the second drug layer, wherein the pre-filled tube is sealed at a proximal end with a sealing cap to create a vacuum inside of the pre-filled tube.

In some embodiments, the pre-filled tube of the third aspect may include one or more of the following features. The first drug layer may be closer to a distal end of the pre-filled tube than the second drug layer. The second drug layer may be closer to a distal of the pre-filled tube than the first drug layer. The anticoagulant may comprise anticoagulant citrate dextrose A (ACD-A), buffered citrate, sodium citrate, citrate phosphate dextrose (CPD), ethylenediaminetetraacetic acid (EDTA), heparin, oxolate, a derivative thereof, or any combination thereof. The anticoagulant may be ACD-A. The coagulating agent may comprise a calcium salt. The calcium salt may be calcium gluconate and/or calcium chloride. The calcium salt may have a concentration of from about 50 mg/mL to about 150 mg/mL, inclusive. The stabilizing agent may be ascorbic acid. The stabilizing agent may have a concentration of from about 400 mg/mL to about 600 mg/mL, inclusive.

A fourth aspect of the present disclosure is directed to a pre-filled tube configured to prepare a platelet rich fibrin matrix (PRFM) layer, wherein the pre-filled tube contains five layers of liquid, gel, or solid material and no more than five layers of liquid, gel, or solid material, the five layers of liquid, gel, or solid material comprising: a first drug layer comprising calcium gluconate and/or calcium chloride in a concentration of about 50 mg/mL to about 150 mg/mL; a second drug layer comprising ascorbic acid in a concentration of from about 400 mg/mL to about 600 mg/mL; a third drug layer comprising an anticoagulant; a first gel layer separating the first drug layer and the second drug layer; and a second gel layer separating the third drug layer from the first drug layer and the second drug layer, wherein the pre-filled tube is sealed at a proximal end with a sealing cap to create a vacuum inside of the pre-filled tube.

A fifth aspect of the present disclosure is directed to a pre-filled tube configured to prepare a platelet rich fibrin matrix (PRFM) layer, wherein the pre-filled tube comprises: a first drug layer comprising a coagulating agent; a second drug layer comprising a stabilizing agent; a third drug layer comprising an anticoagulant; a first gel layer; and a second gel layer, wherein one of the first drug layer and the second drug layer has a distal end that contacts the pre-filled tube and a proximal end that contacts the first gel layer, another of the first drug layer and the second drug layer has a distal end that contacts the first gel layer and a proximal end that contacts the second gel layer, and the third drug layer has a distal end that contacts the second gel layer and a proximal end that contacts a space in the pre-filled tube.

In some embodiments, the pre-filled tube of the fifth aspect may be sealed at a proximal end with a sealing cap to create a vacuum inside of the pre-filled tube.

In some embodiments of the disclosure, the pre-filled tube is a container sized to accommodate a volume of a blood sample of about 29 mL, wherein: (i) the volume of a first drug layer is about 2.0 mL; (ii) the volume of a first gel layer is about 4.3 mL; (iii) the volume of a second drug layer is about 2.0 mL; (iv) the volume of a second gel layer is about 4.3 mL; and (v) the volume of a third drug layer is about 3.2 mL; and wherein: (vi) the first drug layer includes a coagulating agent comprising calcium gluconate and/or calcium chloride, and the second drug layer includes a stabilizer comprising ascorbic acid, wherein a concentration of the calcium gluconate and/or calcium chloride is about 100 mg/mL, and wherein a concentration of the ascorbic acid is about 500 mg/mL; (vii) the first gel and the second are each a thixotropic gel; and (viii) the third drug layer includes an anticoagulant comprising ACD-A.

In some embodiments, the container is sized to accommodate a volume of the blood sample of about 32 mL, wherein: (i) the volume of a first drug layer is about 2.2 mL; (ii) the volume of a first gel layer is about 4.3 mL; (iii) the volume of a second drug layer is about 2.2 mL; (iv) the volume of a second gel layer is about 4.3 mL; and (v) the volume of a third drug layer is about 3.6 mL; and wherein: (vi) the first drug layer includes a coagulating agent comprising calcium gluconate and/or calcium chloride, and the second drug layer includes a stabilizer comprising ascorbic acid, wherein a concentration of the calcium gluconate and/or calcium chloride is about 100 mg/mL, and wherein a concentration of the ascorbic acid is about 500 mg/mL; (vii) the first gel layer and the second gel layer are each a thixotropic gel; and (viii) the third drug layer includes an anticoagulant comprising ACD-A.

In some embodiments: (i) the volume of a first drug layer is from about 0.5 mL to about 4 mL; (ii) the volume of the first gel layer is from about 0.5 mL to about 6 mL; (iii) the volume of the second drug layer is from about 0.5 mL to about 4 mL; (iv) the volume of the second gel layer is from about 0.5 mL to about 6 mL; and (v) the volume of the third drug layer is from about 0.1 mL to about 6 mL; and wherein: (vi) the first drug layer includes a coagulating agent comprising calcium gluconate and/or calcium chloride, and the second drug layer includes a stabilizer comprising ascorbic acid, wherein a concentration of the calcium gluconate and/or calcium chloride is about 100 mg/mL, and wherein a concentration of the ascorbic acid is about 500 mg/mL; (vii) the first gel layer and the second gel layer are each a thixotropic gel; and (viii) the third drug layer includes an anticoagulant comprising ACD-A.

The disclosure features a kit for preparing a PRFM fraction from blood, including: (a) the pre-filled tube or container; and, optionally, one or more of the following: (b) a syringe including a needle; (c) a PRFM dispenser; and (d) a wound dressing.

### Definitions

To facilitate an understanding of this disclosure, a number of terms are defined below.

Terms defined herein have meanings as commonly understood by a person of ordinary skill in the areas relevant to the disclosure. Terms such as “a,” “an,” and “the” are not intended to refer to only a singular entity; but include the general class of which a specific example may be used for illustration. The terminology herein is used to describe specific embodiments of the disclosure, but their usage does not limit the disclosure, except as outlined in the claims.

As used herein, the term “about” refers to a value that is within 10% above or below the value being described.

As used herein, any values provided in a range of values include both the upper and lower bounds, and any values contained within the upper and lower bounds.

“Platelet poor plasma” or “PPP” refers to a blood-derived plasma product from which blood cells (e.g., platelets and red blood cells) have been substantially removed. Greater than 95% RBCs may be removed from the PPP. The PPP may include less than about 0.5% residual hematocrit (e.g., less than about 0.4% residual hematocrit or less than about 0.3% residual hematocrit). The PPP may be separated from a blood sample through centrifugation with one or more separator gels that separates blood components based on density.

“Platelet rich plasma” or “PRP” refers to a blood-derived product when platelets are resuspended in PPP. Due to the presence of the platelets, PRP may be stimulated to release growth factors and other proteins that promote healing. The PRP may have a liquid state and contain little to no fibrin, providing a rapid, short-term release of growth factors and other proteins. The viscosity of the PRP may be from about 1 cP to about 1.3 cP. The PRP may include at least about 2.5×10⁵ platelets/μL (e.g., at least 3×10⁵ platelets/μL, at least 3.5×10⁵ platelets/μL, at least 4×10⁵ platelets/μL, at least 4.5×10⁵ platelets/μL, or at least 5.5×10⁵ platelets/μL).

“Platelet rich fibrin matrix” or “PRFM” refers to a blood-derived product when PRP is activated to initiate a coagulation cascade, converting fibrinogen to fibrin. The PRFM may be activated by the addition of a coagulating agent such as thrombin or a calcium salt (e.g., calcium gluconate and/or calcium chloride). The formation of fibrin may lead to the development of a fibrin matrix, which suspends the platelets, released growth factors, and other proteins that promote healing, allowing for a slower, sustained release of these bioactive molecules over the span of several days (e.g., from 1 day to a week, or time points therebetween). The fibrin matrix may form a gel-like matrix. As such, the PRFM may be used for localized treatments, particularly those needing a composition having a structure (e.g., gel-like matrix). The PRFM may have various degrees of coagulation, including PRFM in a fluid state and/or in a gel state. The viscosity of the PRFM may be about 1 cP to about 1000 cP and may be dependent on the degree of fibrin polymerization and platelet concentration. The PRFM may not be fully coagulated to a gel state when removed from the tube. Accordingly, the PRFM may have a viscous, fluid state that is still capable of being administered or applied with a syringe. For example, the PRFM may be removed from the tube with a syringe in a liquid state having a viscosity less than 100 cP. In some embodiments, the PRFM may be at least partially coagulated in the tube to be more viscous when administered or applied. For example, the PRFM may at least partially be in a gel state when removed. The PRFM may be removed from the tube having a viscosity greater than 100 cP. Once fully formed to a gel-like state (e.g, at the wound site), the PRFM may have a viscosity of up to 1000 cP.

“Treating” or “treatment” refers to the medical management of a patient with the intent that an amelioration, repair, or prevention of an injury or disease, pathological condition, or disorder will result. This term includes active treatment, that is, treatment directed specifically toward improvement of the injury or disease, pathological condition, or disorder, and also includes causal treatment, that is, treatment directed toward removal of the cause of the injury or disease, pathological condition, or disorder. In addition, this term includes palliative treatment, that is, treatment designed for the relief of symptoms rather than the curing of the injury or disease, pathological condition, or disorder; preventive treatment, that is, treatment directed to prevention of the injury or disease, pathological condition, or disorder; and supportive treatment, that is, treatment employed to supplement another specific therapy directed toward the improvement of the injury or disease, pathological condition, or disorder.

### BRIEF DESCRIPTION OF THE DRAWINGS

The following detailed description of the embodiments of the disclosure may be better understood when read in conjunction with the appended drawings. It should be understood, however, that the disclosure is not limited to the precise arrangement and instrumentalities of the embodiments shown in the drawings.

FIGS. 1A-1D illustrate a method of using a first exemplary embodiment of a pre-filled tube, according to the present disclosure.

FIG. 2 illustrates the pre-filled tube of FIG. 1A.

FIGS. 3A-3D illustrate a method of using a second exemplary embodiment of a pre-filled tube, according to the present disclosure.

### DETAILED DESCRIPTION OF THE DRAWINGS

The disclosure provides autologous wound care for a natural, safe, and effective approach for treating chronic and complex wounds. The disclosure may use a patient's own cells and/or tissue to treat wounds, reducing the risk of immune rejection or allergic reactions. The disclosure provides devices, methods, and kits for preparation of platelet rich fibrin matrix (PRFM).

Particularly, the disclosure provides a pre-filled tube containing one or more drug layers separated by one or more gel layers. For example, the present disclosure provides pre-filled tube that include three or five layers of liquid, gel, or solid. The pre-filled tube may minimize handling at the point of care and produce a stabilized platelet gel having an optimal release profile of growth factors (GF). The pre-filled tube may be a centrifuge tube configured to be spun to separate components of the blood. Blood components may then be mixed with one or more of the drug layers to form the PRFM.

The one or more drug layers may include a coagulating agent configured to initiate a coagulation cascade of PRP without fully transitioning the resulting PRFM to a coagulated state.

The coagulating agents (e.g., calcium gluconate and/or calcium chloride) may precondition and/or activate the blood sample, causing insoluble fibrinogen to convert to fibrin, thereby promoting the coagulation cascade. The PRFM fraction may remain in a fluid state that may be readily extracted from the tube to be used for therapeutic and/or cosmetic purposes, for example with a syringe. The one or more drug layers may include a stabilizing agent (e.g., ascorbic acid) to preserve the viscosity of the PRFM and to ease handling of the PRFM. The coagulating agent and the stabilizing agent may mix with platelet poor plasma (PPP) of a blood sample during centrifugation. The PRFM may then be produced by resuspending platelets from the blood sample into the PPP mixed with the coagulating agent and the stabilizing agent. The one or more drug layers may further include an anti-coagulant to regulate the natural clotting pathways of the blood before centrifugation and contact with the coagulating agent. The one or more gel layers may separate the one or more drug layers in the tube to avoid mixing before centrifugation.

The pre-filled tube only requires a single centrifuge spin with all of the mixing performed inside of the centrifuge tube. However, in some embodiments, additional centrifuge spins may be applied if desired. The pre-filled tube reduces the procedural time and the number of steps involved with preparation of PRFM, avoiding the need for external processing and mixing reagents while providing a reproduceable methodology. The pre-filled tube reduces the need for calculations and handling of reagents at the point of care and avoids potential human error. The pre-loaded drug layers may be optimized to promote healing in ulcers (e.g., diabetic foot ulcers, pressure ulcers, or venous leg ulcers), for orthopedic applications (e.g., soft tissue repair, such as tendon or ligament repair, or to improve bone healing), as a hemostatic agent (e.g., a fibrin sealant), and/or for aesthetic and/or dermatologic applications (e.g., facial rejuvenation, hair restoration, scar healing or improvement, and/or treatment of damaged skin and tissue, such as due to burns). Heat (e.g., body heat) may be used to fully activate the coagulation cascade of the resulting PRFM fraction, thereby forming, e.g., a PRFM gel. Additionally or alternatively, an agent or drug could be added to the separated PRFM fraction after centrifugation in order to initiate or expedite the coagulation cascade (e.g., to expedite formation of a PRFM gel).

In some embodiments, the coagulating agent includes a calcium salt, an aluminosilicate, a thrombin, a polyethylene glycol, a derivative thereof, or any combination thereof. In some embodiments, the calcium salt includes calcium gluconate, calcium chloride, calcium saccharate, a derivative thereof, or any combination thereof. In some embodiments, the calcium salt is calcium gluconate and/or calcium chloride. In some embodiments, the first drug layer includes from about 50 mg/mL to about 150 mg/mL (e.g., about 50 mg/mL, about 75 mg/mL, about 100 mg/mL, about 125 mg/mL, or about 150 mg/mL) of calcium gluconate and/or calcium chloride. In some embodiments, the stabilizing agent includes an ascorbic acid, sodium ascorbate, lactate, gluconic acid, and/or citrate. In some embodiments, the second drug layer includes from about 400 mg/mL to about 600 mg/mL (e.g., about 400 mg/mL, about 450 mg/mL, about 500 mg/mL, about 550 mg/mL or about 600 mg/mL) of ascorbic acid.

In some embodiments, the blood sample is centrifuged to separate an RBC fraction and to mix the coagulating agent and the stabilizing agent in the plasma of the blood sample. Platelets of the blood sample may then resuspended into the plasma mixture to form the PRFM. In some embodiments, the first gel layer and the second gel layer intermix to form a single gel layer that separates the plasma from the RBC fraction. In some embodiments, an amount of the coagulating agent, an amount of the stabilizing agent, and an amount of the anticoagulant controls an extent of activation of the platelets to form the PRFM fraction.

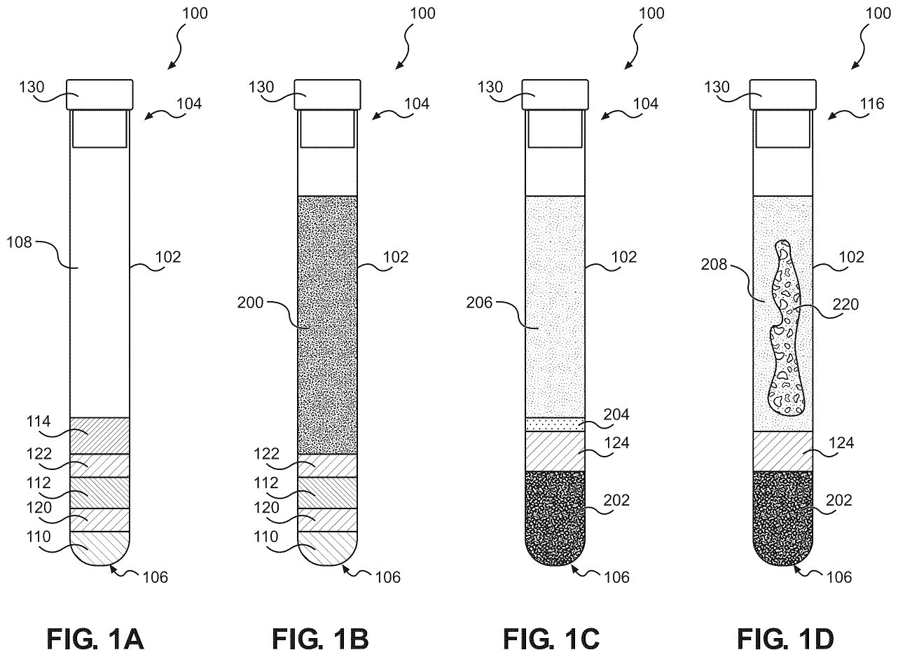

FIGS. 1A-1D illustrate a method of using a first embodiment of a pre-filled container or tube 100, according to the present disclosure. As illustrated in FIG. 1A, the pre-filled tube 100 may comprise a tube 102 with a proximal end 104 and a distal end 106. A sealing cap 130 may be disposed at the proximal end 104 of the tube 102. The tube 102 may contain a plurality of layers of material, such as five layers of material. From the distal end 106 to the proximal end 104, the tube 102 may contain a first drug layer 110, a first gel layer 120, a second drug layer 112, a second gel layer 122, and a third drug layer 114.

At least one of the first drug layer 110 and the second drug layer 112 may include a coagulating agent. At least one of the first drug layer 110, the second drug layer 112, and the third drug layer 114 may include a stabilizing agent. The third drug layer 114 may include an anticoagulant. For example, in some embodiments, the first drug layer 110 may comprise or consist of a coagulating agent, the second drug layer 112 may comprise or consist of a stabilizing agent, and the third drug layer may comprise or consist of an anticoagulant. In some embodiments, the first drug layer 110 may comprise or consist of the stabilizing agent, the second drug layer 112 may comprise or consistent of the coagulating agent, and the third drug layer 112 may comprise or consist of the anticoagulant. In some embodiments, the first drug layer 110 may comprise or consist of a first coagulating agent, the second drug layer 112 may comprise or consist of a second coagulating agent, and the third drug layer 114 may comprise or consist of the anticoagulant. The first coagulating agent and the second coagulating agent may be the same or different. The first gel layer 120 and the second gel layer 122 may comprise a thixotropic gel configured to separate the drug layers 110, 112, 114 before centrifugation. The gel layers 120, 122 may prevent any mixing between the coagulating agent, the stabilizing agent, and the anticoagulant prior to centrifugation. The remaining area within the tube 102 above the third drug layer 114 may be a space 108 to receive a blood sample 200.

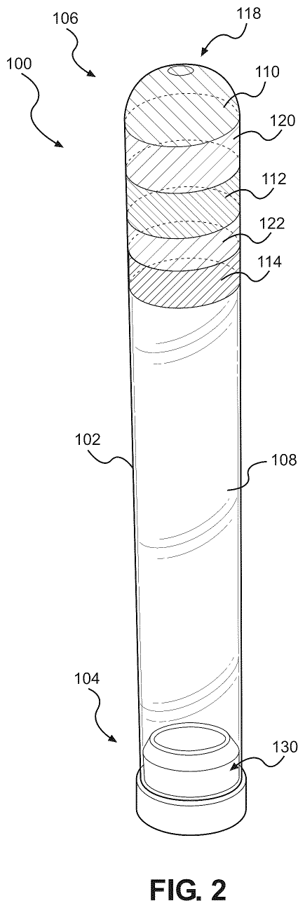

The tube 102 may be prefilled as illustrated in FIG. 1A and ready to be filled with the blood sample 200 by the user. FIG. 2 illustrates an isometric view of the pre-filled tubes 100. As illustrated in FIGS. 1A and 2, the tube 102 may be prefilled with the first drug layer 110, the first gel layer 120, the second drug layer 112, the second gel layer 122, and the third drug layer 114.

Each layer 112-116, 120-122 may remain unperturbed despite the tube 102 being inverted, as illustrated. In some embodiments, the pre-filled tube 100 may be pre-filled with additional layers of liquid, gel, or solid, including bioactive drug layers and/or non-bioactive gel layers. In some embodiments, the pre-filled tube 100 may be pre-filled with only the five layers 110-114, 120-122 of liquid, gel, or solid (without any additional layers of material of liquid, gel, or solid (bioactive or non-bioactive) prior to filling with the blood sample 200).

After pre-filling, the sealing cap 130 (e.g., a stopper) may be inserted into the proximal end 104 of the tube 102 to provide an airtight (e.g., a vacuum) seal inside of the tube 102, as further illustrated. The sealing cap 130 may be configured to provide access to the space 108 inside of the tube 102 without disrupting the seal, for example by puncturing the sealing cap 130 with a needle of a syringe.

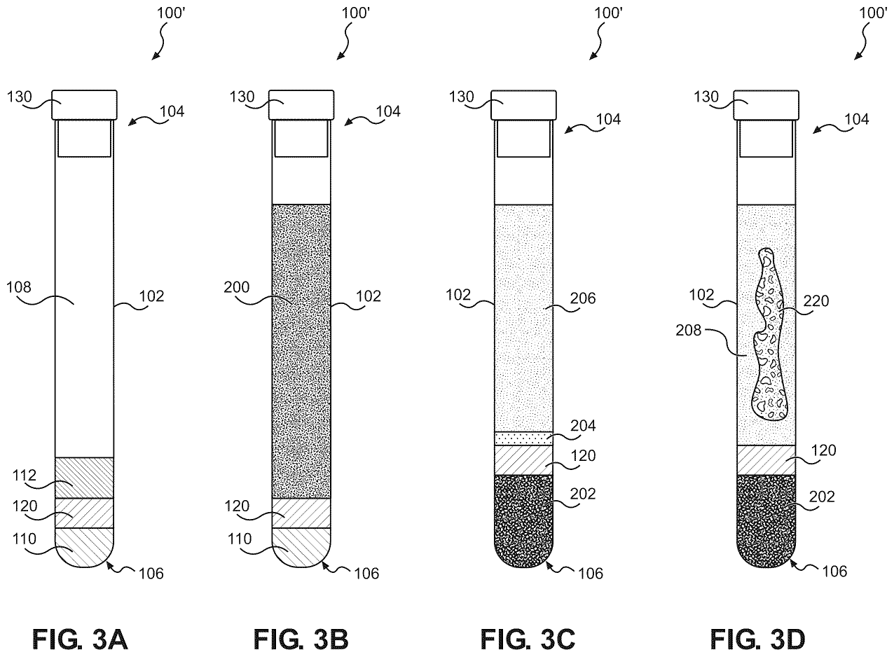

FIGS. 3A-3D illustrate a method of using a second embodiment of the pre-filled tube 100′ without the third drug layer 116 and the second gel layer 122. Thus, FIG. 3A shows an embodiment of the pre-filled tube 100′, in which the tube 102 contains three layers of material. From the distal end 106 to the proximal end 104, the tube 102 may contain the first drug layer 110, the gel layer 120, and the second drug layer 112. The gel layer 120 may separate the first drug layer 110 and the second drug layer 112 before centrifugation. In some embodiments, the first drug layer 110 may comprise or consist of the coagulating agent, and the second drug layer 112 may comprise or consist of the anticoagulant. In some embodiments, the first drug layer 110 may comprise or consist of the coagulating agent, and the second drug layer 112 may comprise or consist of the stabilizing agent. The remaining volume within the tube 100 above the second drug layer 112 is space 108, which may be under vacuum when tube 102 is sealed. In some embodiments, the pre-filled tube 100 may be pre-loaded with additional layers of liquid, gel, or solid, including bioactive drug layers and/or non-bioactive gel layers. In some embodiments, the pre-filled tube 100 may be pre-loaded with only the three layers 110, 112, 120 of liquid, gel, or solid (without any additional layers of material of liquid, gel, or solid (bioactive or non-bioactive) prior to filling with the blood sample 200).

The entire disclosure of the first embodiment of FIGS. 1A-1D is expressly incorporated into the second embodiment of FIGS. 3A-3D for sake of brevity, except when otherwise indicated.

As illustrated in FIGS. 1B, 3B, the space 108 in the tube 102 may be at least partially filled with the blood sample 200. The tube 102 may be filled with the blood sample 200 through a pressure differential from the vacuum of the space 108. For example, a needle assembly (e.g., a butterfly needle, not shown) may be used to transfer the blood sample 200 from a patient to the tube 102. The needle assembly may include a first needle that punctures skin of the patient and a second needle that punctures the sealing cap 130. A tubing may transfer the blood sample 200 from the first needle to the second needle. The second needle may be attached to a tube connector that receives the proximal end 104 of the tube 102 to align the second needle with the sealing cap 130. The size and/or the internal vacuum pressure of the tube 102 may be used to control the input of the blood sample 200, which may also be used to control the volume of PRFM.

As further illustrated, the blood sample 200 may mix with the material of an upper layer (not covered by a gel layer 120, 122). For example, the blood sample 200 may mix with the third drug layer 114 (as illustrated in FIG. 1B) or the second drug layer 112 (as illustrated in FIG. 3B).

The drug third drug layer 114 (FIG. 1A) or the second drug layer 112 (FIG. 3A) may include anticoagulant to prevent natural coagulation to occur when the blood sample 200 is first introduced into the tube 102. The anticoagulant may bind to calcium in the blood sample 200 to prevent the conversion of prothrombin to thrombin. After the blood sample 200 is introduced, the tube 102 may be inverted at least one time to mix the blood 200 with the anticoagulant prior to centrifugation. When initially filled, the gel layer(s) 120, 122 may separate the blood sample 200 from the first drug layer 110 and/or the second drug layer 112 to prevent mixing and/or activation through contact with the coagulating agent.

As illustrated in FIGS. 1C, 3C, the tube 102 may be centrifuged. The method may include a single centrifuging step. The centrifugation may mix and/or separate contents of the tube 102 into layers based on density. The gel layer(s) 120, 122 may liquefy and shift in response to the centrifugal force. The controlled movement of the gel layer(s) 120, 122 enables the mixing of the drug layers 110, 112, 114 with the blood sample 200. The configuration permits the mixing of the drug layers 110, 112, 114 with the plasma of the blood sample 200 through centrifugation, providing a convenient and reliable method for producing the PRFM without the risk of remixing with the other blood components. The mixing of the drug layers 112, 114 with the plasma may initiate a coagulation cascade. The coagulating agent may reverse or neutralize natural coagulants, inducing soluble proteins (e.g., fibrinogen) from the plasma to convert into fibrin to clot.

As illustrated, from the distal end 106 to the proximal end 104, the centrifugation may form a red blood cell (RBC) fraction layer 202, a gel layer 120 or 124, a platelet layer 204, and a plasma mixture (or PPP) layer 206 in the tube 102. Red blood cells of the blood 200 may have a higher density than the remaining components. Thus, during centrifugation, the red blood cells may separate from plasma of the blood 200, pass through the first gel layer 120 and/or the second gel layer 122, and may settle at the distal end 106 of the tube 102 in the RBC fraction layer 202. Platelets of the blood may have a density less than the first gel layer 120 and/or the second gel layer 122 but higher than plasma of the blood 200, the first drug layer 110, and/or the second drug layer 112. Thus, the platelets may separate from the plasma and not pass through the first gel layer 120 and/or the second gel layer 122. The plasma of the blood 200 may mix with the first drug layer 110 and/or the second drug layer 112 to form the plasma mixture layer 206 at the top of the layers. As illustrated in FIG. 1C, the first gel layer 120 and/or the second gel layer 122 may combine to form a gel layer 124 interface between the RBC fraction layer 202 and the platelet layer 204. For example, when the first gel layer 120 and the second gel layer 122 are the same material, the centrifugation may form a single, uniform gel layer 124.

As illustrated in FIGS. 1D, 3D, the platelet layer 204 may be resuspended into the plasma mixture layer 206 to form a PRFM layer 208. For example, the cap 130 may be removed from the proximal end 104 of the tube 102, and a pipette of a syringe may be inserted into the tube.

The syringe may be used to apply fluid pressure to the plasma mixture layer 206 to force the platelet layer 204 into the plasma mixture layer 206 and mix without disturbing the gel layer 120, 124 to keep the RBC layer 202 separated.

The resuspension of the platelets into the plasma mixture layer 206 may place the platelets in a state to further the coagulation cascade. Over time, the PRFM layer 208 may further coagulate and solidify to form a PRFM gel fraction 220. The platelets may be activated when mixed with the plasma mixture layer 206 to release coagulation factors and proteins and to clot to form the PRFM gel fraction 220. The PRFM gel fraction 220 may start forming about 10 minutes after centrifuging and/or resuspending the platelets, depending on the type and/or concentrations of the drugs and/or volume of the blood sample 200.

Without being bound by any theory, the coagulating agent(s) may precondition the blood sample 200 and/or activate the platelets, which causes thrombin to initiate the release of fibrin, thereby promoting the coagulation cascade. The type of coagulating agent(s) may control the extent of activation of the coagulation cascade. The ratio of the amount of the one or more coagulating agents to the amount of the one or more anticoagulants, if present, may control the extent of activation of the PRFM layer 208 (and, accordingly, formation of PRFM). Furthermore, the ratio of the amount of the coagulating agent(s) and, optionally, anticoagulants to the volume of the blood sample 200 may also control the extent of activation of the PRFM layer 208. The amount of coagulating agent(s) may be controlled by the volume of the corresponding drug layer 110, 112 and the concentration of the drug(s) present in the drug layer.

The stabilizing agent may stabilize the PRFM and regulate the activation of the platelets. Without being bound by any theory, the stabilizing agent may maintain the structural integrity of the fibrin matrix. The stabilizing agent may provide tensile strength to the fibrin matrix. The stabilizing agent may further improve healing when applied to a wound by enhancing the stability of extracellular matrix (ECM), particularly for type I and III collagen, during the initial phases of healing.

The PRFM may be removed from the PRFM layer 208 with a biomaterial dispenser (e.g., a syringe and/or forceps) at various degrees of clotting, for example about 1 minute (or less) to about 20 minutes after centrifugation and/or resuspending the platelets. At least a portion of the PRFM layer 208 may be removed from the tube 102 when still in a viscous, liquid state. For example, at least a portion of the PRFM layer 208 may be removed from the tube 102 with a syringe shortly after resuspending the platelets and before substantially forming the PRFM gel fraction 220. For example, the PRFM layer 208 may be removed from the tube 102 within 10 minutes of centrifuging and/or resuspending the platelets. The PRFM layer 208 may then further coagulate at the wound site to form the PRFM gel fraction 220. The present disclosure allows the PRP or PRFM to be mixed quickly (e.g., by reducing transferring and/or mixing of reagents) to allow removal and application of the PRP or PRFM in a liquid state before substantially forming into the PRFM gel fraction 220, which can be performed at the wound site. Additionally or alternatively, the PRFM may be removed from the tube 102 after the PRFM layer 208 begins to solidify into the PRFM gel fraction 220, for example with forceps. For example, the PRFM layer 208 may be removed from the tube 102 after 10 minutes or later following the centrifuging and/or resuspending.

The PRFM may facilitate healing of damaged tissue, promote regeneration of damaged tissue, and/or reduce scarring of the tissue (e.g., skin). For example, the PRFM may be applied to tissue for promoting healing in ulcers (e.g., diabetic foot ulcers, pressure ulcers, or venous leg ulcers), for orthopedic applications (e.g., soft tissue repair (e.g., tendon or ligament repair)), as a hemostatic agent, and/or aesthetic and/or dermatologic applications (e.g., facial rejuvenation, hair restoration, or scar improvement). The PRFM may be used topically (e.g., as a topical wound composition and/or as or with a dressing) or injected (e.g., injectable PRFM solution) into a site for treatment (e.g., a tissue site) or combined with the bone. The PRFM may be used as an autologous treatment with reduced preparation time and/or steps, for example with a single centrifugation and reduced transferring of blood components, for example by mixing the reagents and resuspending the platelets in the tube 102.

The PRFM may also be used with a wound dressing (not shown) to apply over a wound site. In embodiments of wound treatment, the method may include applying the wound dressing over the wound to retain the PRFM at the wound site and/or promote healing. In some embodiments, the PRFM may be applied to the wound before the wound dressing is placed over the wound. In some embodiments, the wound dressing may be placed over the wound before the PRFM is applied to the wound. The wound dressing may include an opening in communication with a tubing, where a syringe filled with the PRFM may connect to a proximal end of the tubing. The syringe may then inject the PRFM through the opening of the wound dressing and onto the wound when the wound dressing is positioned over the wound.

The pre-filled tube 100, 100′ may be a component of a kit for preparing PRFM. The kit may be for cosmetic applications (e.g., hair restoration, facial rejuvenation, scar improvement, or anti-aging treatment), medical applications (e.g., wound (e.g., surgical, traumatic, or burn wounds) and/or tissue healing (e.g., soft tissue, skin, or bone healing) or, e.g., treatment of an injury (e.g., musculoskeletal injuries) or a condition (e.g., osteoarthritis or tendonitis)).

The kit may include the needle assembly (not shown) to transfer the blood sample 200 from the patient to the tube 102. For example, the needle assembly (e.g., a butterfly needle) may include the first needle, the second needle, the tubing, and/or the tube holder. The first needle may be configured to puncture the skin of the patient to withdraw the blood sample 200. The second needle may be configured to puncture the sealing cap 130. The second needle may be attached to the tube connector that receives the proximal end 104 of the tube 102 to align the second needle with the sealing cap 130. The tubing may connect the first needle and the second needle to transfer the blood sample 200 from the patient to the pre-filled tube 100, 100′.

The kit may include one or more syringes and/or needles to resuspend the platelets. The PRFM dispenser may include forceps, an additional tube, a vial, a reservoir, and/or any suitable fluid container configured to hold and dispense a biomaterial. The additional tube may include a syringe. The syringe may be connected to a needle. The needle may be used for insertion into sealing cap 130 to draw the PRP or PRFM from the tube 102. The additional tube may include a male Luer-lock plug disposed at one end of the tube configured to connect to the female Luer-lock threads of proximal region of a connector configured to engage with a proximal end 104 or sealing cap 130 of pre-filled tube 100.

In some embodiments, the PRFM dispenser includes an anticoagulant or a coagulating agent. In some embodiments, the kit further includes a vial including an anticoagulant. In some embodiments, the kit further includes a vial including a coagulating agent. In some embodiments, the kit further includes a tubing, a butterfly needle, a tube holder, and a second syringe, wherein the tube holder is connectable to the sealing cap of the container. In some embodiments, the kit further includes a PRFM storage tube. In some embodiments, the kit further includes a dish for containing a volume of the PRFM fraction for additional processing.

Additionally or alternatively, the kit may include the wound dressing. The wound dressing may be used with PRFM to treat a wound of a subject. The wound dressing may be applied to the subject (e.g., adhered or wrapped around) over the treatment site. The wound dressing may be conformable to the subject to allow wearability. The wound dressing may be sterile. The wound dressing may adhere to the subject.

### Drug Layers 110, 112, 114

The disclosure includes at least one drug layer 110, 112, 114 in the tube 102. The first drug layer 110 and the second drug layer 112 may be the same or different (e.g., each drug layer may include a different drug, a different combination of drugs, or different amounts of the same drugs). The first drug layer 110 and the second drug layer 112 may have the same volume or different volumes. One of the drug layers 112, 114 may have a distal end of that contacts a gel layer 120, 122 and a proximal end that interfaces with the space 108. At least one of the drug layers 112 may have a proximal end and a distal end that each contact a gel layer 120, 122. One of the drug layers 110 may have a proximal end that contacts a gel layer 120, 122 and a distal end that contacts the tube 102. For example, in the embodiment of FIG. 1A, the first drug layer 110 may have a distal end that contacts the distal end 106 of the tube 102. The second drug layer 112 may have a distal end that contacts the first gel layer 120 and a proximal end that contacts the second gel layer 122. The third drug layer 114 may have a distal end that contacts the second gel layer 122 and a proximal end that interfaces with the space 108. Similarly in the embodiment of FIG. 3A, the first drug layer 110 may have a distal end that contacts the distal end 106 of the tube 102. The second drug layer 112 may have a distal end that contacts the gel layer 120 and a proximal end that interfaces with the space 108.

Upon introduction of the blood sample 200 to the pre-filled tube 100, the blood sample 200 may contact the proximal-most drug layer 112, 114 prior to centrifugation. At least one of the drug layers 110, 112 may be distal of the gel layer(s) 120, 122, such that, upon filling of the tube with a blood sample, the gel layer(s) 120, 122 separates the blood sample 200 from the drug layer(s) 110, 112. During centrifugation, the drug layer(s) 110, 112, 114 mix with plasma of the blood sample 200.

At least one of the first drug layer 110 and/or the second drug layer 112 may comprise the coagulating agent. Preferably, the coagulating agent is separated from the blood sample 200 by the gel layer(s) 120, 122 when initially introduced into the tube 102 (e.g., FIG. 1B, 3B) to prevent premature coagulation. The coagulating agent(s) may be any agent capable of promoting coagulation of the blood sample 200 or its respective components during separation (e.g., the coagulating agent may be capable of promoting coagulation of the PPP and/or PRP). Examples of a coagulating agent that may be included in a drug layer include a calcium salt, an aluminosilicate (e.g., kaolin), thrombin, polyethylene glycol, or a combination thereof. The calcium salt may be, e.g., calcium chloride, calcium gluconate, calcium saccharate, a derivative thereof, or combinations thereof. One or more of the drug layers may include a plurality of coagulating agents.

For example, the coagulating agent may be calcium gluconate and/or calcium chloride.

The calcium gluconate and/or calcium chloride may be present in at least one of the drug layers 110, 112 at a concentration of from about 50 mg/mL to about 150 mg/mL (e.g., about 50 mg/mL, about 75 mg/mL, about 100 mg/mL, about 125 mg/mL or about 150 mg/mL). Calcium gluconate and/or calcium chloride may be included in at least one of the drug layers 110, 112 at a concentration that pre-activates the platelets without producing full coagulation (e.g., without causing the formation of a gel).

At least one of the first drug layer 110, the second drug layer 112, and the third drug layer 114 may comprise the stabilizing agent. The stabilizing agent may be ascorbic acid, sodium ascorbate, lactate, gluconic acid, and/or citrate. The stabilizing agent may be ascorbic acid present in at least one of the drug layers 110, 112, 114 at a concentration of from about 100 mg/mL to about 900 mg/mL, such as from about 400 mg/mL to about 600 mg/mL (e.g., about 400 mg/mL to about 500 mg/mL, about 500 mg/mL to about 600 mg/mL, about 450 mg/mL to about 550 mg/mL, e.g., about 400 mg/mL, about 450 mg/mL, about 500 mg/mL, about 550 mg/mL, or about 600 mg/mL, inclusive).

The first drug layer 110 and the second drug layer 112 may each have a volume of from about 0.5 mL to about 4 mL (e.g., from about 0.5 mL to about 1.0 mL, from about 0.5 mL to about 2.0 mL, from about 0.5 mL to about 3.0 mL, from about 0.5 mL to about 4.0 mL, from about 1.0 mL to about 2.0 mL, from about 1.0 mL to about 3.0 mL, from about 1.0 mL to about 4.0 mL, from about 2.0 mL to about 3.0 mL, from about 2.0 mL to about 4.0 mL, from about 3.0 mL to about 4.0 mL, e.g., about 0.5 mL, about 0.7 mL, about 1 mL, about 1.3 mL, about 1.5 mL, about 2.0 mL, about 2.2 mL, about 2.5 mL, about 3.0 mL, about 3.5 mL, or about 4.0 mL).

The first drug layer 110 may have a density of from about 1.0 g/mL to about 1.4 g/mL (e.g., about 1.0 g/mL, about 1.1 g/mL, about 1.2 g/mL, about 1.3 g/mL, or about 1.4 g/mL). The second drug layer 112 may have a density of from about 1.0 g/mL to about 1.4 g/mL (e.g., about 1.0 g/mL, about 1.1 g/mL, about 1.2 g/mL, about 1.3 g/mL, or about 1.4 g/mL).

The ratio of the volume of the first drug layer 110 to the volume of the blood sample 200 may be from about 1:4 to about 1:30 (e.g., about 1:4 to about 1:10, about 1:4 to about 1:15, about 1:13 to about 1:16, about 1:10 to about 1:20, about 1:20 to about 1:30, e.g., about 1:4, about 1:5, about 1:6, about 1:7, about 1:8, about 1:9, about 1:10, about 1:11, about 1:12, about 1:13, about 1:14, or about 1:15, about 1:16, about 1:17, about 1:18, about 1:19, about 1:20, about 1:21, about 1:22, about 1:23, about 1:24, or about 1:25, about 1:26, about 1:27, about 1:28, about 1:29, or about 1:30).

The ratio of the volume of the second drug layer 112 to the volume of the blood sample 200 may be from about 1:4 to about 1:30 (e.g., about 1:4 to about 1:10, about 1:4 to about 1:15, about 1:13 to about 1:16, about 1:10 to about 1:20, about 1:20 to about 1:30, e.g., about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, or about 1:25, about 1:26, about 1:27, about 1:28, about 1:29, or about 1:30).

The ratio of the combined volume of the first drug layer 110 and the second drug layer 112 to the volume of the blood sample 200 may be from 1:2 to about 1:15 (e.g., about 1:2 to about 1:10, about 1:4 to about 1:15, about 1:12 to about 1:15, about 1:10 to about 1:15, about 1:6 to about 1:10, e.g., about 1:2, about 1:3, about 1:4, about 1:5, about 1:6, about 1:7, about 1:8, about 1:9, about 1:10, about 1:11, about 1:12, about 1:13, about 1:14, or about 1:15).

When the blood sample 200 is centrifuged in the pre-filled tube 100, the ratio of the volume of the first drug layer 110 to the volume of the PRFM layer 208 may be from about 1:2 to about 1:15 (e.g., about 1:2 to about 1:5, about 1:4 to about 1:10, about 1:4 to about 1:15, about 1:7 to about 1:9, about 1:10 to about 1:15, e.g., about 1:2, about 1:3, about 1:4, about 1:5, about 1:6, about 1:7, about 1:8, about 1:9, about 1:10, about 1:11, about 1:12, about 1:13, about 1:14, or about 1:15).

The ratio of the volume of the second drug layer 112 to the volume of the PRFM layer 208 may be from about 1:2 to about 1:15 (e.g., about 1:2 to about 1:5, about 1:4 to about 1:10, about 1:4 to about 1:15, about 1:7 to about 1:9, about 1:10 to about 1:15, e.g., about 1:2, about 1:3, about 1:4, about 1:5, about 1:6, about 1:7, about 1:8, about 1:9, about 1:10, about 1:11, about 1:12, about 1:13, about 1:14, or about 1:15).

The ratio of the combined volume of the first drug layer 110 and the second drug layer 112 to the volume of the PRFM layer 208 may be from 1:1 to about 1:8 (e.g., about 1:1 to about 1:3, about 1:2 to about 1:6, about 1:4 to about 1:8, about 1:2 to about 1:4, about 1:5 to about 1:8, about 1:6 to about 1:8, e.g., about 1:1, about 1:2, about 1:3, about 1:4, about 1:5, about 1:6, about 1:7, or about 1:8).

The ratio of the volume of the first drug layer 110 to the volume of the second drug layer 112 may be about 1:1. The first drug layer 110 and second drug layer 112 may each have a volume of from about 1% to about 15% (e.g., about 1% to about 12%, about 1% to about 8%, about 1% to about 4%, about 2% to about 10%, about 2% to about 7%, about 4% to about 12%, about 4% to about 8%, about 6% to about 8%, about 6% to about 15%, about 10% to about 15%, e.g., about 1%, about 2%, about 3%, about 4%, about 5%, about 6%, about 7%, about 8%, about 9%, about 10%, about 11%, about 12%, about 13%, about 14%, or about 15%) of the volume of the PRP preparation tube.

The third drug layer 114 (FIG. 1A) or the second drug layer 112 (FIG. 3A) may comprise the anticoagulant (“anticoagulant layer”). The anticoagulant may provide more time for the user to use the PRFM prior to its full coagulation (e.g., formation of a solid gel). Alternatively, an anticoagulant layer may not be included, such as when the blood sample 200 drugs that inhibit the clotting or coagulation of PRP (e.g., blood thinners), as an anticoagulant may further elongate the process for pre-conditioning and/or activating the PRP. The type of anticoagulant(s) may control the extent of inhibition of the coagulation cascade. The ratio of the amount of the one or more coagulating agents to the amount of the one or more anticoagulants may also control the extent of activation of the PRP fraction. Furthermore, the ratio of the amount of each of the coagulating agent(s) and the anticoagulant(s) to the volume of the blood sample may also control the extent of activation of the PRP fraction. The amount of anticoagulant may be controlled by the volume of the anticoagulant layer and the concentration of the anticoagulant present in the anticoagulant layer.

The anticoagulant may be any agent capable of inhibiting, reducing, or down-regulating coagulation of the blood or its respective components during separation (e.g., the anticoagulant may inhibit or reduce a coagulation cascade in the blood or in either of the PRP fraction and the RBC fraction). The distal end of the anticoagulant layer may contact a gel layer, and the proximal end of the anticoagulant layer may interface the space 108 in the tube 102 such that, upon filling of the tube with the blood sample 200, the anticoagulant layer may contact the blood sample 200. For example, in some embodiments, the third drug layer 114 may comprise the anticoagulant (e.g., FIG. 1A) and contact the second gel layer 122. In some embodiments, the second drug layer 112 may comprise the anticoagulant (e.g., FIG. 3A) and contact the first gel layer 120. Upon introduction of the blood sample 200 into the tube 102, the proximal end of the anticoagulant layer may interface with the blood sample 200 prior to centrifugation. Prior to centrifugation, the tube may be inverted at least one time (e.g., at least 5 times, at least 6 times, or at least 7 times, e.g., 5 to 10 times, 7 to 10 times, e.g., 1 time, 2 time, 3 times, 4 times, 5 times, 6 times, 7 times, 8 times, 9 times, or 10 times) to mix the anticoagulant layer with the blood sample 200.

The anticoagulant may include buffered citrate, sodium citrate, citrate phosphate dextrose (CPD), ethylenediaminetetraacetic acid (EDTA), heparin, oxolate, anticoagulant citrate dextrose A (ACD-A), any derivative thereof, and combinations thereof. The anticoagulant may be ACD-A. ACD-A is composed of citric acid, sodium dextrose, and dextrose. ACD-A may include about 7 mg/mL citric acid, about 22 mg/mL sodium citrate, and about 24.5 mg/mL dextrose. The tube 102 may include a single anticoagulant layer that includes a plurality of anticoagulants. The anticoagulant layer may have a density of about 1.0 g/mL.

The anticoagulant layer may have a volume of from about 0.1 mL to about 6 mL (e.g., from about 0.1 mL to about 0.5 mL, from about 0.5 mL to about 1.0 mL, from about 0.5 mL to about 2.0 mL, from about 0.5 mL to about 3.0 mL, from about 0.5 mL to about 4.0 mL, from about 0.5 mL to about 5.0 mL, from about 0.5 mL to about 6.0 mL, from about 1.0 mL to about 2.0 mL, from about 1.0 mL to about 3.0 mL, from about 1.0 mL to about 4.0 mL, from about 1.0 mL to about 5.0 mL, from about 1.0 mL to about 6.0 mL, from about 1.5 mL to about 2.0 mL, from about 2.0 mL to about 3.0 mL, from about 2.0 mL to about 4.0 mL, from about 2.0 mL to about 5.0 mL, from about 2.0 mL to about 6.0 mL, from about 3.0 mL to about 4.0 mL, about 3.0 mL to about 5.0 mL, from about 3.0 mL to about 6.0 mL, from about 4.0 mL to about 4.5 mL, from about 4.0 mL to about 5.0 mL, from about 4.0 mL to about 6.0 mL, from about 5.0 mL to about 6.0 mL, e.g., about 0.1 mL, about 0.2 mL, about 0.3 mL, about 0.4 mL, about 0.5 mL, about 0.7 mL, about 1 mL, about 1.3 mL, about 1.5 mL, about 1.9 mL, about 2.0 mL, about 2.5 mL, about 3.0 mL, about 3.5 mL, about 4.0 mL, about 4.25 mL, about 4.3 mL, about 4.5 mL, about 5.0 mL, about 5.5. mL, or about 6.0 mL). In some embodiments, the tube 102 does not include an anticoagulant layer.

The ratio of the volume of the anticoagulant layer to the volume of the blood sample 200 may be from about 1:5 to about 1:100 (e.g., about 1:5 to about 1:10, about 1:5 to about 1:15, about 1:10 to about 1:20, about 1:5 to about 1:50, about 1:50 to about 1:100, about 1:25 to about 1:50, about 1:50 to about 1:75, e.g., about 1:100, about 1:95, about 1:90, about 1:85, about 1:80, about 1:75, about 1:70, about 1:65, about 1:60, about 1:55, about 1:50, about 1:45, about 1:40, about 1:35, about 1:30, about 1:25, about 1:20, about 1:15, about 1:10, about 1:9, about 1:8, about 1:7, about 1:6, or about 1:5).

When centrifuged, the ratio of the volume of the anticoagulant layer to the volume of the PRFM layer 208 may be from about 1:3 to about 1:65 (e.g., about 1:3 to about 1:5, about 1:3 to about 1:10, about 1:10 to about 1:20, about 1:5 to about 1:55, about 1:25 to about 1:50, about 1:30 to about 1:55, about 1:40 to about 1:65, e.g., about 1:65, about 1:60, 1:55, about 1:50, about 1:45, about 1:40, about 1:35, about 1:30, about 1:25, about 1:20, about 1:15, about 1:10, about 1:9, about 1:8, about 1:7, about 1:6, about 1:5, about 1:4, or about 1:3).

The anticoagulant layer may have a volume of from about 1% to about 15% (e.g., about 1% to about 12%, about 1% to about 8%, about 1% to about 4%, about 2% to about 10%, about 2% to about 7%, about 4% to about 12%, about 4% to about 8%, about 6% to about 8%, about 6% to about 15%, about 10% to about 15%, e.g., about 1%, about 2%, about 3%, about 4%, about 5%, about 6%, about 7%, about 8%, about 9%, about 10%, about 11%, about 12%, about 13%, about 14%, or about 15%) of the volume of the tube 102.

The ratio of the volume of the anticoagulant to the volume of the other drug layers may be from about 1:3 to about 5:1 (e.g., about 1:3 to about 1:2, about 1:3 to about 1:1, about 1:2 to about 1:1, about 1:1 to about 2:1, about 2:1 to about 3:1, about 3:1 to about 4:1, about 4:1 to about 5:1, e.g., about 1:3, about 1:2, about 1:1, about 2:1, about 3:1, about 4:1, or about 5:1).

The ratio of the volume of the anticoagulant layer to the volume of the gel layer 120, 122 may be from about 1:5 to about 3:1 (e.g., about 1:5 to about 1:3, about 1:4 to about 1:3, about 1:4 to about 1:2, about 1:4 to about 1:1, about 1:3 to about 1:1, about 1:3 to about 1:2, about 1:2 to about 1:1, about 1:1 to about 2:1, about 2:1 to about 3:1, e.g., about 1:5, about 1:4, about 1:3, about 1:2, about 1:1, about 2:1, or about 3:1).

When the pre-filled tube 102 includes five layers (see, e.g., FIG. 1A), the ratio of the volume of the third drug layer 114 to the volume of the first drug layer 110 or the second drug layer 112 may be from about 1:3 to about 5:1 (e.g., about 1:3 to about 1:2, about 1:3 to about 1:1, about 1:2 to about 1:1, about 1:1 to about 2:1, about 2:1 to about 3:1, about 3:1 to about 4:1, about 4:1 to about 5:1, e.g., about 1:3, about 1:2, about 1:1, about 2:1, about 3:1, about 4:1, or about 5:1).

In some embodiments, the volume of the first drug layer 110 and the second drug layer 112 is the same. In some embodiments, the volume of the first drug layer 110 is different from the volume of the third drug layer 114. In some embodiments, the volume of the first drug layer 110 and the volume of the third drug layer 114 are each from about 0.5 mL to about 4.0 mL (e.g., from about 0.5 mL to about 1.0 mL, from about 0.5 mL to about 2.0 mL, from about 0.5 mL to about 3.0 mL, from about 0.5 mL to about 4.0 mL, from about 1.0 mL to about 2.0 mL, from about 1.0 mL to about 3.0 mL, from about 1.0 mL to about 4.0 mL, from about 2.0 mL to about 3.0 mL, from about 2.0 mL to about 4.0 mL, from about 3.0 mL to about 4.0 mL, e.g., about 0.5 mL, about 0.7 mL, about 1 mL, about 1.3 mL, about 1.5 mL, about 2.0 mL, about 2.2 mL, about 2.5 mL, about 3.0 mL, about 3.5 mL, or about 4.0 mL).

The pH of each of the one or more drug layer(s) 110, 112, 114 may be from about 5.5 to about 8.5 (e.g., about 5.6 to about 6.6, about 6 to about 8.2, about 6 to about 8.5, e.g., about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7., about 7.8, about 7.9, about 8, about 8.1, about 8.2, about 8.3, about 8.4, or about 8.5).

The pH of the one or more drug layer(s) 110, 112 containing calcium gluconate and/or calcium chloride as the coagulating agent may be from about 6 to about 8.5 (e.g., about 6 to about 6.5, about 6.5 to about 7, about 7 to about 7.5, about 7.5 to about 8, about 8 to about 8.5, e.g., about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7., about 7.8, about 7.9, about 8, about 8.1, about 8.2, about 8.3, about 8.4, or about 8.5).

The pH of the one or more drug layer(s) 110, 112, 114 containing ascorbic may be from about 5.6 to about 6.6 (e.g., about 5.6 to about 6.1, about 6.1 to about 6.6, e.g., about 5.5, about 5.6, about 5.7, about 5.8, about 5.9, about 6, about 6.1, about 6.2, about 6.3, about 6.4, about 6.5, or about 6.6). The pH of the one or more drug layer(s) containing an anticoagulant may be from about 4.5 to about 5.5 (e.g., about 4.5 to about 5, about 5 to about 5.5, e.g., about 4.5, about 4.6, about 4.7, about 4.8, about 4.9, about 5, about 5.1, about 5.2, about 5.3, about 5.4, or about 5.5).

The weighted median pH is calculated by first determining the total volume of the solution (e.g. the total volume of all drug layers in the tube 102). For each component (e.g., layer) of the solution, the contribution of the overall pH is calculated by weighing its pH range endpoints (e.g., the minimum pH and the maximum pH) against the component's (e.g., layer's) volume fraction. Accordingly, the following equations may be used to calculate the weighted pH, where i represents the layer:

$$Weighted pH ( minimum ) = \sum ( \frac{{Volume}_{i}}{Total Volume} \times {pH}_{min , i} )$$

$$Weighted pH ( maximum ) = \sum ( \frac{{Volume}_{i}}{Total Volume} \times {pH}_{max , i} )$$

Each of the weighted pHs for the minimum pH is added together to provide the lower endpoint of the weighted pH range of the solution. Each of the weighted pHs for the maximum pH is added together to provide the upper endpoint of the weighted pH range of the solution. The weighted median pH is calculated in the same manner, using the median pH for each of the components (e.g., layers). Alternatively, the weighted median pH may be calculated by calculation of the median using the upper and lower endpoints of the weighted pH range.

The weighted pH of the combination of the one or more drug layer(s) 110, 112, 114 may be from about 4.5 to about 8 (e.g., about 4.5 to about 5, about 5.0 to about 5.5, about 5.5 to about 6.0, about 6.0 to about 6.5, about 6.5 to about 7, about 7 to about 7.5, about 7.5 to about 8, about 6 to about 7.5, about 5 to about 6.5, about 5 to about 6, about 6 to about 7, about 4.5 to about 6, about 5 to about 7. e.g., about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, or about 8). The weighted pH of each of the one or more drug layer(s) may be from about 1.2 to about 4.5 (e.g., about 1.2 to about 1.5, about 1.5 to about 2, about 2 to about 2.5, about 2.5 to about 3, about 3 to about 3.5, about 3.5 to about 4, about 4 to about 4.5, e.g., about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, or about 4.5).

The weighted pH of the anticoagulant layer may be from about 1.5 to 4 (e.g., about 1.5 to about 2, about 2 to about 2.5, about 2.5 to about 3, about 3 to about 3.5, about 3.5 to about 4, e.g., about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, or about 4).

### Gel Layers 120, 122

The gel layers 120, 122 may maintain separation between the one or more drug layers 110, 112, 114 prior to centrifugation of the tube 102. The gel layer 120, 122 may maintain separation of the blood sample 200 from the drug layers 112, 114 after filling the tube 102. The gel layer 120, 122 may further maintain separation of the RBC layer 202 after centrifugation.

The first gel layer 120 and/or the second gel layer 122 becomes less viscous when agitated or subjected to shear stress but returns to a more viscous state when at rest. During centrifugation, the gel layer 120, 122 allows the passage of RBCs through and beyond the gel, creating a distinct layer 120, 124 that separates the RBCs from the platelets and the plasma. The first gel layer 120 and/or the second gel layer 122 may be a thixotropic gel. In some embodiments, the first gel 120 and the second gel 122 are each a thixotropic gel.

The first gel layer 120 and the second gel layer 122 may be the same. Alternatively, the first gel layer 120 and the second gel layer 122 may be different (e.g., the gel layer may include two different gel components or the gel layers may have different thixotropic properties). The first gel layer 120 and the second gel layer 122 may intermix during centrifugation to form the gel layer 124 after centrifugation.

The first gel layer 120 and/or the second gel layer 122 may be a thixotrpic gel. The thixotropic gel may include petroleum hydrocarbon resin; tris(2-elthylhexyl) benzene-1,2,4-tricarboxylate; silicon dioxide; silane, dichlorodimethyl-, reaction products with silica; polyolefin oligomer; polyacrylic acid; and/or polyamide.

In some embodiments, the first gel layer 120 and/or the second gel layer 122 may have a density of from about 1.03 g/mL to about 1.7 g/mL (e.g., about 1.03 mg/mL, about 1.4 mg/mL, about 1.5 mg/mL, about 1.6 mg/mL, or about 1.7 mg/mL).

The first gel layer 120 and/or the second gel layer 122 may have a mass of from 0.5 g to about 12.0 g (e.g., from about 1 g to about 2 g, from about 2 g to about 3 g, from about 3 g to about 4 g, from about 3 g to about 5 g, from about 5 g to about 10 g, from about 8 g to about 10 g, from about 8 g to about 12 g, from about 3 g to about 9 g, e.g., about 1 g, about 1.5 g, about 2 g, about 2.5 g, about 3 g, about 3.5 g, about 4 g, about 4.5 g, about 5 g, about 5.5 g, about 6 g, about 6.5 g, about 7 g, about 7.5 g, about 8 g, about 8.5 g, about 9 g, about 9.5 g, about 10 g, about 10.5 g, about 11 g, about 11.5 g, or about 12 g).

The first gel layer 120 and/or the second gel layer 124 may a volume of from about 0.5 mL to about 6 mL (e.g., from about 0.5 mL to about 1.0 mL, from about 0.5 mL to about 2.0 mL, from about 0.5 mL to about 3.0 mL, from about 0.5 mL to about 4.0 mL, from about 0.5 mL to about 5.0 mL, from about 1.0 mL to about 2.0 mL, from about 1.0 mL to about 3.0 mL, from about 1.0 mL to about 4.0 mL, from about 1.0 mL to about 5.0 mL, from about 1.0 mL to about 6.0 mL, from about 2.0 mL to about 3.0 mL, from about 2.0 mL to about 4.0 mL, from about 2.0 mL to about 5.0 mL, from about 2.0 mL to about 6.0 mL, from about 3.0 mL to about 4.0 mL, about 3.0 mL to about 5.0 mL, from about 3.0 mL to about 6.0 mL, from about 4.0 mL to about 5.0 mL, from about 4.0 mL to about 6.0 mL, from about 5.0 mL to about 6.0 mL, e.g., about 0.5 mL, about 0.7 mL, about 1 mL, about 1.5 mL, about 2.0 mL, about 2.5 mL, about 3.0 mL, about 3.5 mL, about 4.0 mL, about 4.5 mL, about 5.0 mL, about 5.5. mL, or about 6.0 mL).

In some embodiments, the first gel layer 120 and/or the second gel layer 124 may have a combined volume of about 1 mL to about 12 mL (e.g., from about 1 mL to about 2 mL, from about 2 mL to about 3 mL, from about 3 mL to about 4 mL, from about 3 mL to about 5 mL, from about 5 mL to about 10 mL, from about 8 mL to about 10 mL, from about 8 mL to about 12 mL, from about 3 mL to about 9 mL, e.g., about 1 mL, about 1.5 mL, about 2 mL, about 2.5 mL, about 3 mL, about 3.5 mL, about 4 mL, about 4.5 mL, about 5 mL, about 5.5 mL, about 6 mL, about 6.5 mL, about 7 mL, about 7.5 mL, about 8 mL, about 8.5 mL, about 9 mL, about 9.5 mL, about 10 mL, about 10.5 mL, about 11 mL, about 11.5 mL, or about 12 mL). In some embodiments, the volume of the first gel layer 120 and the volume of the second gel layer 122 is the same, or the volume of the first gel layer is different than the volume of the second gel layer.

The ratio of volume of the gel layer(s) 120, 122 to the volume of the blood sample 200 may be from about 1:2 to about 1:10 (e.g., about 1:2 to about 1:4, about 1:2 to about 1:6, about 1:2 to about 1:7, about 1:4 to about 1:6, about 1:5 to about 1:7, about 1:5 to about 1:10, about 1:7 to about 1:10, e.g., about 1:2, about 1:3, about 1:4, about 1:5, about 1:6, about 1:7, about 1:8, about 1:9, or about 1:10).

The gel layer(s) 120, 122 may have a volume of from about 3% to about 25% (e.g., about 1% to about 12%, about 1% to about 8%, about 1% to about 4%, about 2% to about 10%, about 2% to about 7%, about 4% to about 12%, about 4% to about 8%, about 6% to about 8%, about 6% to about 15%, about 10% to about 15%, about 5% to about 20%, about 10% to about 20%, about 15% to about 20%, about 20 to about 25%, e.g., about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%) of the volume of the tube 102.

In embodiments with a single gel layer 120 (see, e.g., FIG. 3A), the ratio of the volume of the first drug layer 110 or the second drug layer 112 to the gel layer 120 may be from about 1:7 to about 2:1 (e.g., about 1:7 to about 1:5, about 1:7 to about 1:6, about 1:7 to about 1:1, about 1:7 to about 1:4, about 1:5 to about 1:4, about 1:5 to about 1:3, about 1:5 to about 1:2, about 1:4 to about 1:2, about 1:4 to about 1:3, about 1:4 to about 1:1, about 1:3 to about 1:2, about 1:3 to about 1:1, about 1:2 to about 1:1, about 1:1 to about 2:1, e.g., about 2:1, about 1:1, about 1:2, about 1:3, about 1:4, about 1:5, about 1:6, or about 1:7).

In such embodiments with the single gel layer 120 (see, e.g., FIG. 3A), the first drug layer 110 may have a volume of about 0.5 mL to about 4 mL (e.g., from about 0.5 mL to about 1.0 mL, from about 0.5 mL to about 2.0 mL, from about 0.5 mL to about 3.0 mL, from about 0.5 mL to about 4.0 mL, from about 1.0 mL to about 2.0 mL, from about 1.0 mL to about 3.0 mL, from about 1.0 mL to about 4.0 mL, from about 2.0 mL to about 3.0 mL, from about 2.0 mL to about 4.0 mL, from about 3.0 mL to about 4.0 mL, e.g., about 0.5 mL, about 0.7 mL, about 1 mL, about 1.3 mL, about 1.5 mL, about 2.0 mL, about 2.2 mL, about 2.5 mL, about 3.0 mL, about 3.5 mL, or about 4.0 mL). The first drug layer 110 may have a density of about 1.0 g/mL to about 1.4 g/mL (e.g., about 1.0 g/mL, about 1.1 g/mL, about 1.2 g/mL, about 1.3 g/mL, or about 1.4 g/mL). When tube blood sample 200 is added to the pre-filled tube 100, the ratio of the volume of the first drug layer 110 to the volume of the blood sample 200 may be from about 1:4 to about 1:30 (e.g., about 1:4 to about 1:10, about 1:4 to about 1:15, about 1:13 to about 1:16, about 1:10 to about 1:20, about 1:20 to about 1:30, e.g., about 1:4, about 1:5, about 1:6, about 1:7, about 1:8, about 1:9, about 1:10, about 1:11, about 1:12, about 1:13, about 1:14, or about 1:15, about 1:16, about 1:17, about 1:18, about 1:19, about 1:20, about 1:21, about 1:22, about 1:23, about 1:24, or about 1:25, about 1:26, about 1:27, about 1:28, about 1:29, or about 1:30).

### Tube 102

The pre-filled tube 100, 100′ (referred collectively as 100, except when otherwise indicated) may be designed as a container defined by the tube 102.

The tube 102 may be any size or shape that is suitable for centrifugation (e.g., a centrifugation tube or a plasma preparation tube).

The tube 102 may have a volume of about 5 mL to about 60 mL (e.g., from about 5 mL to about 10 mL, from about 5 mL to about 15 mL, from about 15 mL to about 20 mL, from about 5 mL to about 40 mL, from about 9 mL to about 36 mL, from about 10 mL to about 40 mL, from about 10 mL to about 50 mL, from about 20 mL to 40 mL, from about 30 mL to about 35 mL, from about 30 mL to 40 mL, from about 20 mL to 30 mL, from about 50 mL to about 55 mL, or from about 50 mL to about 60 mL, e.g., about 5 mL, about 10 mL, about 15 mL, about 16 mL, about 20 mL, about 25 mL, about 30 mL, about 32 mL, about 35 mL, about 40 mL, about 45 mL, about 50 mL, about 54 mL, about 55 mL, or about 60 mL).

The tube 102 may be a 16 by 125 mm tube, a 20×125 mm tube, or a 29×115 mm tube.

The volume of the space (e.g., air) 108 within the tube 102 may be from about 4 mL to about 50 mL (e.g., about 5 mL to about 10 mL, about 10 mL to about 20 mL, about 20 mL to about 30 mL, about 30 mL to about 40 mL, about 40 mL to about 50 mL, about 10 mL to about 30 mL, about 15 mL to about 45 mL, e.g., about 5 mL, about 10 mL, about 11 mL, about 15 mL, about 20 mL, about 22 mL, about 25 mL, about 30 mL, about 34 mL, about 35 mL, about 40 mL, about 45 mL, or about 50 mL). The sealing cap may displace from about 0.5 mL to about 4 mL (e.g., from about 0.5 mL to about 1.0 mL, from about 0.5 mL to about 2.0 mL, from about 0.5 mL to about 3.0 mL, from about 0.5 mL to about 4.0 mL, from about 1.0 mL to about 2.0 mL, from about 1.0 mL to about 3.0 mL, from about 1.0 mL to about 4.0 mL, from about 2.0 mL to about 3.0 mL, from about 2.0 mL to about 4.0 mL, from about 3.0 mL to about 4.0 mL, e.g., about 0.5 mL, about 0.7 mL, about 1 mL, about 1.3 mL, about 1.5 mL, about 2.0 mL, about 2.2 mL, about 2.5 mL, about 3.0 mL, about 3.5 mL, or about 4.0 mL) of the volume of the tube 102.

The tube 102 may be configured to draw from about 7 mL to about 22 mL of the blood sample 200 (e.g., from about 7 mL to about 10 mL, about 10 mL to about 18 mL, about 8 mL to about 12 mL, from about 18 mL to about 20 mL, from about 19 mL to about 20 mL, from about 20 mL to about 22 mL, e.g., about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 21 mL, or about 22 mL of the blood sample 200) may have a pressure of between about negative 90 kPa and about negative 100 kPa (e.g., about negative 90 kPa, about negative 91 kPa, about negative 92 kPa, about negative 93 kPa, about negative 94 kPa, about negative 95 kPa, about negative 96 kPa, about negative 97 kPa, about negative 98 kPa, about negative 99 kPa, or about negative 100 kPa, e.g., about negative 90 to about negative 95 kPa or about negative 95 kPa to negative 100 kPa).

Negative pressure or vacuum in the tube 102 may be maintained by the sealing cap 130.

The negative pressure of the tube 102 may be from about negative 50 kPa to about negative 101 kPa (e.g., about negative 80 kPa to about negative 100 kPa, e.g., about negative 50 kPa, about negative 55 kPa, about negative 60 kPa, about negative 65 kPa, about negative 70 kPa, about negative 75 kPa, about negative 80 kPa, about negative 85 kPa, about negative 86 kPa, about negative 87 kPa, about negative 88 kPa, about negative 89 kPa, about negative 90 kPa, about negative 91 kPa, about negative 92 kPa, about negative 93 kPa, about negative 94 kPa, about negative 95 kPa, about negative 96 kPa, about negative 97 kPa, about negative 98 kPa, about negative 99 kPa, about negative 100 kPa, or about negative 101 kPa).

The interior of the tube 102 may include a coating (e.g., a silicone coating).

The tube 102 may be configured to draw from about 10 mL to about 36 mL of the blood sample 200 (e.g., from about 10 mL to about 30 mL, from about 10 mL to about 20 mL, from about 15 mL to about 30 mL, from about 15 mL to about 20 mL, from about 30 mL to about 33 mL, from about 33 mL to about 36 mL, from about 32 mL to about 36 mL, from about 34 mL to about 36 mL, from about 30 mL to about 32 mL, or from about 35 mL to about 36 mL, e.g., about 12 mL, about 13 mL, about 14 mL, about 15 mL, about 16 mL, about 17 mL, about 18 mL, about 19 mL, about 20 mL, about 21 mL, about 22 mL, about 23 mL, about 24 mL, about 25 mL, about 26 mL, about 27 mL, about 28 mL, about 29 mL, about 30 mL, about 31 mL, about 32 mL, about 33 mL, about 34 mL, about 35 mL, or about 36 mL of the blood sample 200) may have a negative pressure of between negative 80 kPa and negative 90 kPa (e.g., about negative 80 kPa, about negative 81 kPa, about negative 82 kPa, about negative 83 kPa, about negative 84 kPa, about negative 85 kPa, about negative 86 kPa, about negative 87 kPa, about negative 88 kPa, about negative 89 kPa, or about negative 90 kPa, e.g., about negative 80 to about negative 85 kPa or about negative 85 kPa to negative 90 kPa).

The tube 102 may be configured to draw from about 14 mL to about 40 mL of the blood sample 200 (e.g., from about 14 mL to about 20 mL, from about 15 mL to about 30 mL, from about 15 mL to about 25 mL, from about 25 mL to about 30 mL, from about 20 mL to about 30 mL, from about 31 mL to about 35 mL, from about 35 mL to about 38 mL, from about 38 mL to about 40 mL, from about 34 mL to about 35 mL, from about 34 mL to about 36 mL, from about 34 mL to about 38 mL, from about 36 mL to about 40 mL, e.g., about 14 mL, about 15 mL, about 16 mL, about 17 mL, about 18 mL, about 19 mL, about 20 mL, about 21 mL, about 22 mL, about 23 mL, about 24 mL, about 25 mL, about 26 mL, about 27 mL, about 28 mL, about 29 mL, about 30 mL, about 31 mL, about 32 mL, about 33 mL, about 34 mL, about 35 mL, about 36 mL, about 37 mL, about 38 mL, about 39 mL, or about 40 mL of the blood sample 200) may have a negative pressure of between negative 90 kPa and negative 100 kPa (e.g., about negative 90 kPa, about negative 91 kPa, about negative 92 kPa, about negative 93 kPa, about negative 94 kPa, about negative 95 kPa, about negative 96 kPa, about negative 97 kPa, about negative 98 kPa, about negative 99 kPa, or about negative 100 kPa, e.g., about negative 90 to about negative 95 kPa or about negative 95 kPa to negative 100 kPa).

In some embodiments, the tube 102 further contains the blood sample 200. In some embodiments, the tube 102 includes a volume of from about 5 mL to about 60 mL, and a ratio of the volume of the first gel layer 120 to a volume of the blood sample 200 is from about 1:4 to about 1:20 (e.g., about 1:4 to about 1:8, about 1:4 to about 1:12, about 1:4 to about 1:16, about 1:6 to about 1:8, about 1:6 to about 1:10, about 1:6 to about 1:14, about 1:6 to about 1:20, about 1:10 to about 1:14, about 1:8 to about 1:16, about 1:16 to about 1:20, e.g., about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, or about 1:20).

In some embodiments, the tube 102 includes a volume of from about 5 mL to about 60 mL, and a ratio of the volume of the second gel layer 122 to the volume of the blood sample 200 is from about 1:4 to about 1:20 (e.g., about 1:4 to about 1:8, about 1:4 to about 1:12, about 1:4 to about 1:16, about 1:6 to about 1:8, about 1:6 to about 1:10, about 1:6 to about 1:14, about 1:6 to about 1:20, about 1:10 to about 1:14, about 1:8 to about 1:16, about 1:16 to about 1:20, e.g., about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, or about 1:20).

In some embodiments, the tube 102 includes a volume of from about 5 mL to about 60 mL, and a ratio of a combined volume of the first gel layer 120 and the second gel layer 122 and the volume of the blood sample 200 is from about 1:2 to about 1:10 (e.g., about 1:2 to about 1:4, about 1:2 to about 1:6, about 1:2 to about 1:7, about 1:4 to about 1:6, about 1:5 to about 1:7, about 1:5 to about 1:10, about 1:7 to about 1:10, e.g., about 1:2, about 1:3, about 1:4, about 1:5, about 1:6, about 1:7, about 1:8, about 1:9, or about 1:10).

In some embodiments, the tube 102 may include a volume of from about 5 mL to about 60 mL, and a ratio of the volume of the first drug layer 110 to the volume of the blood sample 200 is from about 1:4 to about 1:30 (e.g., about 1:4 to about 1:10, about 1:4 to about 1:15, about 1:13 to about 1:16, about 1:10 to about 1:20, about 1:20 to about 1:30, e.g., about 1:4, about 1:5, about 1:6, about 1:7, about 1:8, about 1:9, about 1:10, about 1:11, about 1:12, about 1:13, about 1:14, or about 1:15, about 1:16, about 1:17, about 1:18, about 1:19, about 1:20, about 1:21, about 1:22, about 1:23, about 1:24, or about 1:25, about 1:26, about 1:27, about 1:28, about 1:29, or about 1:30).

In some embodiments, the tube 102 may include a volume of from about 5 mL to about 60 mL, and a ratio of the volume of the second drug layer 112 to the volume of the blood sample 200 is from about 1:4 to about 1:30 (e.g., about 1:4 to about 1:10, about 1:4 to about 1:15, about 1:13 to about 1:16, about 1:10 to about 1:20, about 1:20 to about 1:30, e.g., about 1:4, about 1:5, about 1:6, about 1:7, about 1:8, about 1:9, about 1:10, about 1:11, about 1:12, about 1:13, about 1:14, or about 1:15, about 1:16, about 1:17, about 1:18, about 1:19, about 1:20, about 1:21, about 1:22, about 1:23, about 1:24, or about 1:25, about 1:26, about 1:27, about 1:28, about 1:29, or about 1:30).

In some embodiments, the tube 102 includes a volume of from about 5 mL to about 60 mL, and a ratio of a combined volume of the first drug layer 110 and the second drug layer 112 to the volume of the blood sample 200 is from about 1:2 to about 1:15 (e.g., about 1:2 to about 1:10, about 1:4 to about 1:15, about 1:12 to about 1:15, about 1:10 to about 1:15, about 1:6 to about 1:10, e.g., about 1:2, about 1:3, about 1:4, about 1:5, about 1:6, about 1:7, about 1:8, about 1:9, about 1:10, about 1:11, about 1:12, about 1:13, about 1:14, or about 1:15).

In some embodiments, the tube 102 may include a volume of from about 5 mL to about 60 mL, and a ratio of the volume of the third drug layer 114 to the volume of the blood sample 200 is from about 1:5 to about 1:100 (e.g., about 1:5 to about 1:10, about 1:5 to about 1:15, about 1:10 to about 1:20, about 1:5 to about 1:50, about 1:50 to about 1:100, about 1:25 to about 1:50, about 1:50 to about 1:75, e.g., about 1:100, about 1:95, about 1:90, about 1:85, about 1:80, about 1:75, about 1:70, about 1:65, about 1:60, about 1:55, about 1:50, about 1:45, about 1:40, about 1:35, about 1:30, about 1:25, about 1:20, about 1:15, about 1:10, about 1:9, about 1:8, about 1:7, about 1:6, or about 1:5).

In some embodiments, the tube 102 may include a volume of from about 5 mL to about 60 mL, and a ratio of a combined volume of the first drug layer 110, the second drug layer 112, and the third drug layer 114 to the volume of the blood sample 200 is from about 1:1 to about 1:10 (e.g., about 1:2 to about 1:10, about 1:4 to about 1:8, about 1:7 to about 1:10, about 1:2 to about 1:8, about 1:1 to about 1:5, e.g., about 1:1, about 1:2, about 1:3, about 1:4, about 1:5, about 1:6, about 1:7, about 1:8, about 1:9, or about 1:10).

The tube 102 may be a 16×125 mm tube, a 20×125 mm tube, or a 29×115 mm tube.

The tube 102 may be any commercially available tube for the preparation of platelet rich plasma.

The sealing cap 130 may be at the proximal end of the tube 102. The sealing cap 130 may be configured for insertion into the proximal end of the tube in order to seal the tube. For example, the sealing cap may be configured to provide an airtight seal, such that the tube may be placed under vacuum pressure. The sealing cap 130 may also be made from a pierceable material, such as a thermoplastic elastomer (TPE), such as rubber (e.g., bromo butyl rubber), that may be easily pierced, e.g., with a needle and/or syringe.

The sealing cap 130 may be pierced multiple times throughout the preparation of PRFM without it being removed from the tube 102 (e.g., by syringe injection or other known methods).

The needle and/or syringe may be configured to inject the blood sample 200 into the tube 102. For example, a syringe may be connected to another needle (e.g., a butterfly needle), e.g., by way of tubing, for receiving a blood sample as the blood is drawn from a subject. In this example, a tube holder may be equipped with the pre-filled tube 100 for compatibility with the additional components (e.g., needle, tubing, and/or syringe) for drawing blood, such that, as the blood sample 200, it is transferred directly into the tube 102. The sealing cap 130 may also be pierced (e.g., by a needle) for removal of the PFRM after centrifugation.

### Process

The volume of the blood sample 200 added to the pre-filled tube 100 may be from about 2 mL to about 45 mL (e.g., about 2 mL to about 9 mL, about 5 mL to about 10 mL, about 5 mL to about 20 mL, about 5 mL to about 30 mL, about 5 mL to about 40 mL, about 10 mL to about 40 mL, about 20 mL to about 40 mL, about 30 mL to about 40 mL, about 10 mL to about 30 mL, about 30 mL to about 45 mL, about 20 mL to about 30 mL, about 15 mL to about 30 mL, about 10 mL to about 45 mL, e.g., about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 21 mL, about 22 mL, about 23 mL, about 24 mL, about 25 mL, about 26 mL, about 27 mL, about 28 mL, about 29 mL, about 30 mL, about 31 mL, about 32 mL, about 33 mL, about 34 mL, about 35 mL, about 36 mL, about 37 mL, about 38 mL, about 39 mL, about 40 mL, about 41 mL, about 42 mL, about 43 mL, about 44 mL, or about 45 mL).

The centrifugation may be at an acceleration of about 1000 g to about 4500 g (e.g., about 1200 g to about 3500 g, about 1220 g to about 3000 g, about 1220 g to about 2500 g, about 1220 g to about 1500 g, about 1220 g to about 1250 g, about 1500 g to about 3500 g, about 1500 g to about 3000 g, about 1500 g to about 1750 g, about 1750 g to about 2500 g, about 1750 g to about 2250 g, about 1750 g to about 2000 g, about 2000 g to about 2500 g, about 2250 g to about 2500 g, e.g., about 1220 g, about 1200 g, about 1300 g, about 1400 g, about 1500 g, about 1600 g, about 1700 g, about 1800 g, about 1900 g, about 2000 g, about 2100 g, about 2200 g, about 2300 g, about 2400 g, or about 2500 g).

The centrifugation may be for a time between about 3 minutes to about 12 minutes (e.g., about 3 minutes to about 10 minutes, about 5 minutes to about 8 minutes, about 6 minutes to about 12 minutes, about 6 minutes to 10 minutes, about 8 minutes to 12 minutes, about 8 minutes to 10 minutes, about 10 minutes to about 12 minutes or, e.g., about 5 minutes, about 6 minutes, about 7 minutes, about 8 minutes, about 9 minutes, about 10 minutes, about 11 minutes, or about 12 minutes).

### PRFM Layer 208

The PRFM layer 208 may be about 45% to about 65% (e.g., about 45%, about 50%, about 55%, about 60%, or about 65%) of the volume of the blood sample 200. The percentage of PRP layer 208 in the blood sample 200 of the subject may be subject-dependent.

The PRFM layer 208 prepared in the tube 102 may have a volume from about 1 mL to about 30 mL (e.g., about 1 mL to about 5 mL, about 5 mL to about 10 mL, about 10 mL to about 15 mL, about 15 mL to about 20 mL, about 20 mL to about 25 mL, about 25 mL to about 30 mL, about 5 mL to about 20 mL, about 10 mL to about 20 mL, about 15 mL to about 25 mL, e.g., about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 21 mL, about 22 mL, about 23 mL, about 24 mL, or about 25 mL).

In some embodiments, the volume of the PRFM layer 208 may be from 1 mL to about 8 mL (e.g., from about 1 mL to about 4 mL, from about 3 mL to about 7 mL, or from about 4 mL to about 6 mL, e.g., about 1 mL, about 2 mL, about 3 mL, about 4 mL, about 5 mL, about 6 mL, about 7 mL, or about 8 mL) when the tube 102 is configured to draw from about 2 mL to about 12 mL (e.g., about 2 mL to about 10 mL, about 2 mL to about 8 mL, about 2 mL to about 4 mL, about 4 mL to about 8 mL, about 4 mL to about 12 mL, about 8 mL to about 10 mL, about 10 mL to about 12 mL, e.g., about 2 mL, about 3 mL, about 4 mL, about 5 mL, about 6 mL, about 7 mL, about 8 mL, about 9 mL, about 10 mL, about 11 mL, or about 12 mL of the blood sample 200).

In some embodiments, the volume of the PRFM layer 208 may be from about 3 mL to about 13 mL (e.g., from about 3 mL to about 8 mL, from about 8 mL to about 12 mL, or from about 9 mL to about 11 mL, e.g., about 3 mL, about 4 mL, about 5 mL, about 6 mL, about 7 mL, about 8 mL, about 9 mL, about 10 mL, about 11 mL, about 12 mL, or about 13 mL) when pre-filled tube 100 100 is configured to draw from about 7 mL to about 22 mL (e.g., from about 7 mL to about 10 mL, about 10 mL to about 18 mL, about 8 mL to about 12 mL, from about 18 mL to about 20 mL, from about 19 mL to about 20 mL, from about 20 mL to about 22 mL, e.g., about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 21 mL, or about 22 mL of the blood sample 200).

In some embodiments, the volume of the PRFM layer 208 may be from about 6 mL to about 20 mL (e.g., from about 6 mL to about 13 mL, from about 13 mL to about 17 mL, from about 16 mL to about 20 mL, or from about 14 mL to about 16 mL, e.g., about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, or about 20 mL) when pre-filled tube 100 100 is configured to draw from about 12 mL to about 36 mL (e.g., from about 30 mL to about 33 mL, from about 33 mL to about 36 mL, from about 32 mL to about 36 mL, from about 34 mL to about 36 mL, from about 30 mL to about 32 mL, or from about 35 mL to about 36 mL, e.g., about 30 mL, about 31 mL, about 32 mL, about 33 mL, about 34 mL, about 35 mL, or about 36 mL).

In some embodiments, the volume of the PRFM layer 208 may be from about 7 mL to about 23 mL (e.g., from about 7 mL to about 12 mL, from about 10 mL to about 15 mL, from about 15 mL to about 20 mL, from about 18 mL to about 22 mL, from about 20 mL to about 23 mL, or from about 19 mL to about 21 mL, e.g., about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 21 mL, about 22 mL, or about 23 mL) when pre-filled tube 100 is configured to draw from about 14 mL to about 40 mL (e.g., from about 14 mL to about 20 mL, from about 15 mL to about 30 mL, from about 15 mL to about 25 mL, from about 25 mL to about 30 mL, from about 20 mL to about 30 mL, from about 31 mL to about 35 mL, from about 35 mL to about 38 mL, from about 38 mL to about 40 mL, from about 34 mL to about 35 mL, from about 34 mL to about 36 mL, from about 34 mL to about 38 mL, from about 36 mL to about 40 mL, e.g., about 14 mL, about 15 mL, about 16 mL, about 17 mL, about 18 mL, about 19 mL, about 20 mL, about 21 mL, about 22 mL, about 23 mL, about 24 mL, about 25 mL, about 26 mL, about 27 mL, about 28 mL, about 29 mL, about 30 mL, about 31 mL, about 32 mL, about 33 mL, about 34 mL, about 35 mL, about 36 mL, about 37 mL, about 38 mL, about 39 mL, or about 40 mL of the blood sample 200).

The viscosity of the PRFM layer 208 may vary depending on the stage of coagulation. At least a portion of the PRFM layer 208 may be removed from the tube 102 with a viscosity of from about 1 cP to about 1000 cP. In some embodiments, the at least the portion of the PRFM layer 208 may be removed from the tube 102 in a fluid state, with a viscosity less than or equal to 100 cP. In some embodiments, the at least the portion of the PRFM layer 208 may be removed from the tube 102 in a gel state, with a viscosity greater than or equal to 100 cP.

The PRFM layer 208 may include less than about 0.3% residual hematocrit. In some embodiments, wherein the PRFM layer 208 includes at least 2.5×10⁵ platelets/μL. In some embodiments, the PRFM layer 208 includes a concentration of calcium gluconate and/or calcium chloride of from about 3 mg/mL to about 20 mg/mL (e.g., about 3 mg/mL to about 6 mg/mL, about 3 mg/mL to about 12 mg/mL, about 6 mg/mL to about 12 mg/mL, about 12 mg/mL to about 20 mg/mL, about 12 mg/mL to about 15 mg/mL, about 10 mg/mL to about 15 mg/mL, about 15 mg/mL to about 20 mg/mL, e.g., about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL).

In some embodiments, the PRFM layer 208 may include a concentration of ascorbic acid of from about 20 mg/mL to about 75 mg/mL (e.g., about 20 mg/mL to about 30 mg/mL, about 25 mg/mL to about 50 mg/mL, about 45 mg/mL to about 55 mg/mL, about 45 mg/mL to about 65 mg/mL, about 50 mg/mL to about 60 mg/mL, about 60 mg/mL to about 65 mg/mL, about 65 mg/mL to about 75 mg/mL, e.g., about 20 mg/mL, about 21 mg/mL, about 22 mg/mL, about 23 mg/mL, about 24 mg/mL, about 25 mg/mL, about 27 mg/mL, about 28 mg/mL, about 29 mg/mL, about 30 mg/mL, about 32 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 47 mg/mL, about 50 mg/mL, about 52 mg/mL, about 55 mg/mL, about 57 mg/mL, about 60 mg/mL, about 61 mg/mL, about 62 mg/mL, about 63 mg/mL, about 64 mg/mL, about 65 mg/mL, about 67 mg/mL, about 70 mg/mL, about 72 mg/mL, or about 75 mg/mL).

### Example 1

TABLE 1
Contents of the pre-filled tube 100 configured to draw
from about 2 ml to about 12 ml of the blood sample 200

| | | | Volume and/or |
|---|---|---|---|
| Item | Description | Size | Mass |
| Tube 102 | PRP tube, clear, | 16 x 125 mm | 16 mL |
| | silicone-coated | | |
| First drug | Calcium | — | 0.7 mL |
| layer 110 | gluconate and/or | | |
| | calcium | | |
| | chloride, USP 10%, | | |
| | 100 mg/mL | | |
| First gel | Thixotropic | — | 0.75 g (about 0.7 |
| layer 120 | separation serum | | mL) |
| | gel, non-sterile | | |
| Second drug | Ascorbic acid | — | 0.7 mL |
| layer 112 | USP, 500 | | |
| | mg/mL | | |
| Second gel | Thixotropic | — | 0.75 g (about 0.7 |
| layer 122 | separation serum | | mL) |
| | gel, non-sterile | | |
| Third drug | Anticoagulant | — | 1.1 mL |
| layer 114 | citrate dextrose | | |
| | “A” (ADC-A) | | |
| Sealing cap | Tube stopper, | 16 mm | — |
| 130 | butyl rubber | | |

The tube 102 of the pre-filled tube 100 may be configured to draw from about 2 mL to about 12 mL of the blood sample 200 (e.g., about 2 mL to about 10 mL, about 2 mL to about 8 mL, about 2 mL to about 4 mL, about 4 mL to about 8 mL, about 4 mL to about 12 mL, about 8 mL to about 10 mL, about 10 mL to about 12 mL, e.g., about 2 mL, about 3 mL, about 4 mL, about 5 mL, about 6 mL, about 7 mL, about 8 mL, about 9 mL, about 10 mL, about 11 mL, or about 12 mL of the blood sample 200) from a subject.

Table 1 describes the content of each of the five layers in the pre-filled tube 100 of the disclosure configured to draw from about 2 mL to about 12 mL of the blood sample 200, along with the size and materials of the sealing cap 130 and tube 102. The first drug layer 110 and the second drug layer 112 may each have a volume of about 0.7 mL. The first drug layer 110 may include about 100 mg/mL calcium gluconate and/or calcium chloride as a coagulating agent. The second drug layer 112 may include ascorbic acid at a concentration of about 500 mg/mL. As such, first drug layer 110 may include about 70 mg of calcium gluconate and/or calcium chloride, and the second drug layer 112 may include about 350 mg of ascorbic acid. The first gel layer 120 and the second gel layer 122 may each include a non-sterile, thixotropic separation serum gel and may each have a volume of about 0.7 mL, or, equivalently, a mass of about 0.75 g.

The third drug layer 114 may be an anticoagulant including ACD-A and may have a volume of about 1.1 mL. ACD-A may include about 7 mg/mL citric acid, about 22 mg/mL sodium citrate, and about 24.5 mg/mL dextrose. With this, the third drug layer 114 may include about 9 mg of citric acid, about 24.2 mg of sodium citrate, and about 27 mg of dextrose. The tube 102 may be a clear, silicone-coated PRP tube with a size of 16 mm×125 mm. The volume of the tube 102 may be 16 mL. The sealing cap 130 may be a butyl rubber tube stopper that has a size (e.g., diameter) of 16 mm. The pressure of the tube 102 may be about negative 95 kPa. The volume of the space 108 may be about 11 mL. The sealing cap 130 may displace about 1 mL of the volume of the tube 102. The ratio of the volume of the blood sample 200 to the combined volume of the first drug layer 110, the second drug layer 112, and the third drug layer 114 may about 4:1.

### Example 2

TABLE 2
Contents of the pre-filled tube 100 configured to draw
from about 7 mL to about 22 mL of the blood sample 200.

| | | | Volume and/or |
|---|---|---|---|
| Component | Description | Size | Mass |
| Tube 102 | PRP tube, clear, | 20 x 125 mm | 32 mL |
| | silicone-coated | | |
| First drug | Calcium | — | 1.3 mL |
| layer 110 | gluconate and/or | | |
| | calcium | | |
| | chloride, USP 10%, | | |
| | 100 mg/mL | | |
| First gel | Thixotropic | — | 2.0 g (about 1.9 |
| layer 120 | separation serum | | mL) |
| | gel, non-sterile | | |
| Second drug | Ascorbic acid | — | 1.3 mL |
| layer 112 | USP, 500 | | |
| | mg/mL | | |
| Second gel | Thixotropic | — | 2.0 g (about 1.9 |
| layer 122 | separation serum | | mL) |
| | gel, non-sterile | | |
| Third drug | Anticoagulant | — | 2.2 mL |
| layer 114 | citrate dextrose | | |
| | “A” (ADC-A) | | |
| Sealing cap | Tube stopper, | 20 mm | — |
| 130 | butyl rubber | | |

The tube 102 of the pre-filled tube 100 may be configured to draw from about 7 mL to about 22 mL of the blood sample 200 (e.g., from about 7 mL to about 10 mL, about 10 mL to about 18 mL, about 8 mL to about 12 mL, from about 18 mL to about 20 mL, from about 19 mL to about 20 mL, from about 20 mL to about 22 mL, e.g., about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 21 mL, or about 22 mL of blood) from a subject. As little as 2.0 mL of the blood sample 200 may be collected (e.g., if the vein collapses during the blood draw).

Table 2 describes the content of each of the five layers in the pre-filled tube 100 of the disclosure configured to draw from about 7 mL to about 22 mL of the blood sample 200 along with the size and materials of the sealing cap 130 and the tube 102. The first drug layer 110 and second drug layer 112 may each have a volume of about 1.3 mL. The first drug layer 110 may include about 100 mg/mL calcium gluconate and/or calcium chloride as a coagulating agent. The second drug layer 112 may include about 500 mg/mL ascorbic acid as a stabilizing agent. As such, first drug layer 110 may include about 130 mg of calcium gluconate and/or calcium chloride, and second drug layer 112 may include about 650 mg of ascorbic acid. The first gel layer 120 and the second gel layer 122 may each have a volume of about 1.9 mL, or, equivalently, a mass of about 2.0 g. The third drug layer 114 may include an anticoagulant of ACD-A and may have a volume of about 2.2 mL. ACD-A may include about 7 mg/mL citric acid, about 22 mg/mL sodium citrate, and about 24.5 mg/mL dextrose. With this, the third drug layer 114 may include about 15 mg of citric acid, about 48 mg of sodium citrate, and about 54 mg of dextrose. The volume of the tube 102 may be about 32 mL. The tube 102 may be a clear, silicone-coated PRP tube with a size of 20 mm×125 mm. The sealing cap 130 may be a butyl rubber tube stopper that has a size (e.g., diameter) of 20 mm. The pressure of the tube 102 may be about negative 95 kPa. The volume of the space 140 may be about 22 mL. The sealing cap 130 may displace about 1.9 mL of the volume of the tube 102. The ratio of the volume of the blood to the combined volume of first drug layer 110, second drug layer 112, and the third drug layer 114 is about 4:1.

### Example 3

TABLE 3
Contents of the pre-filled tube 100 configured to draw from
about 12 mL to about 36 mL of the blood sample 200.

| | | | Volume and/or |
|---|---|---|---|
| Item | Description | Size | Mass |
| Tube 102 | PRP tube, clear, | 29 x 115 mm | 54 mL |
| | silicone-coated | | |
| First drug | Calcium | — | 2.0 mL |
| layer 110 | gluconate and/or | | |
| | calcium | | |
| | chloride, USP 10%, | | |
| | 100 mg/mL | | |
| First gel | Thixotropic | — | 4.5 g (about 4.3 |
| layer 120 | separation serum | | mL) |
| | gel, non-sterile | | |
| Second drug | Ascorbic acid | — | 2.0 mL |
| layer 112 | USP, 500 | | |
| | mg/mL | | |
| Second gel | Thixotropic | — | 4.5 g (about 4.3 |
| layer 122 | separation serum | | mL) |
| | gel, non-sterile | | |
| Third drug | Anticoagulant | — | 3.2 mL |
| layer 114 | citrate dextrose | | |
| | “A” (ADC-A) | | |
| Sealing cap | Tube stopper, | 29 mm | — |
| 130 | butyl rubber | | |

The tube 102 of the pre-filled tube 100 may be configured to draw from about 12 mL to about 36 mL of the blood sample 200 (e.g., from about 12 mL to about 30 mL, from about 12 mL to about 20 mL, from about 15 mL to about 30 mL, from about 15 mL to about 20 mL, from about 30 mL to about 33 mL, from about 33 mL to about 36 mL, from about 32 mL to about 36 mL, from about 34 mL to about 36 mL, from about 30 mL to about 32 mL, or from about 35 mL to about 36 mL, e.g., about 12 mL, about 13 mL, about 14 mL, about 15 mL, about 16 mL, about 17 mL, about 18 mL, about 19 mL, about 20 mL, about 21 mL, about 22 mL, about 23 mL, about 24 mL, about 25 mL, about 26 mL, about 27 mL, about 28 mL, about 29 mL, about 30 mL, about 31 mL, about 32 mL, about 33 mL, about 34 mL, about 35 mL, or about 36 mL of the blood sample 200) from a subject. As little as 2.0 mL of the blood sample 200 may be collected (e.g., if the vein collapses during the blood draw).

Table 3 describes the content of each of the five layers in the pre-filled tube 100 of the disclosure configured to draw from about 12 mL to about 36 mL of the blood sample 200, along with the size and materials of the sealing cap 130 and tube 102. The first drug layer 110 and the second drug layer 112 may each have a volume of about 2.0 mL. The first drug layer 110 may include about 100 mg/mL calcium gluconate and/or calcium chloride as a coagulating agent. The second drug layer 112 may include about 500 mg/mL ascorbic acid as a stabilizing agent. As such, first drug layer 110 may include about 200 mg of calcium gluconate and/or calcium chloride, and second drug layer 112 may include about 1000 mg of ascorbic acid. The first gel layer 120 and the second gel layer 122 may each have a volume of about 4.3 mL, or, equivalently, a mass of about 4.5 g. The third drug layer 114 may include ACD-A as an anticoagulant and may have a volume of about 3.2 mL. ACD-A may include about 7 mg/mL citric acid, about 22 mg/mL sodium citrate, and about 24.5 mg/mL dextrose. With this, the third drug layer 114 may include about 22 mg of citric acid, about 70 mg of sodium citrate, and about 78 mg of dextrose. The volume of the tube 102 may be about 54 mL. The tube 102 may be a clear, silicone-coated PRP tube with a size of 29 mm×115 mm. The sealing cap 130 may be a butyl rubber tube stopper that has a size (e.g., diameter) of 29 mm. The pressure of the tube 102 may be about negative 85 kPa. The volume of the space 140 may be about 35 mL. The sealing cap 130 may displace about 3.5 mL of the volume of the tube 102. The ratio of the volume of the blood to the combined volume of first drug layer 110, second drug layer 112, and the third drug layer 114 is about 4:1.

### Example 4

TABLE 4
Contents of the pre-filled tube 100 configured to draw
from about 14 mL to about 40 mL of the blood sample
200 (e.g., about 31.6 mL of the blood sample 200).

| | | | Volume and/or |
|---|---|---|---|
| Item | Description | Size | Mass |
| Tube 102 | PRP tube, clear, | 29 x 115 mm | 54 mL |
| | silicone-coated | | |
| First drug | Calcium | — | 2.2 mL |
| layer 110 | gluconate and/or | | |
| | calcium | | |
| | chloride, USP 10%, | | |
| | 100 mg/mL | | |
| First gel | Thixotropic | — | 4.5 g (about 4.3 |
| layer 120 | separation serum | | mL) |
| | gel, non-sterile | | |
| Second drug | Ascorbic acid | — | 2.2 mL |
| layer 112 | USP, 500 | | |
| | mg/mL | | |
| Second gel | Thixotropic | — | 4.5 g (about 4.3 |
| layer 122 | separation serum | | mL) |
| | gel, non-sterile | | |
| Third drug | Anticoagulant | — | 3.6 mL |
| layer 114 | citrate dextrose | | |
| | “A” (ADC-A) | | |
| Sealing cap | Tube stopper, | 29 mm | — |
| 130 | butyl rubber | | |

The tube 102 of the pre-filled tube 100 may be configured to draw from about 14 mL to about 40 mL of the blood sample 200 (e.g., from about 14 mL to about 20 mL, from about 15 mL to about 30 mL, from about 15 mL to about 25 mL, from about 25 mL to about 30 mL, from about 20 mL to about 30 mL, from about 31 mL to about 35 mL, from about 35 mL to about 38 mL, from about 38 mL to about 40 mL, from about 34 mL to about 35 mL, from about 34 mL to about 36 mL, from about 34 mL to about 38 mL, from about 36 mL to about 40 mL, e.g., about 14 mL, about 15 mL, about 16 mL, about 17 mL, about 18 mL, about 19 mL, about 20 mL, about 21 mL, about 22 mL, about 23 mL, about 24 mL, about 25 mL, about 26 mL, about 27 mL, about 28 mL, about 29 mL, about 30 mL, about 31 mL, about 32 mL, about 33 mL, about 34 mL, about 35 mL, about 36 mL, about 37 mL, about 38 mL, about 39 mL, or about 40 mL of the blood sample 200 from a subject. As little as 2.0 mL of the blood sample 200 may be collected (e.g., if the vein collapses during the blood draw).

Table 4 describes the content of each of the five layers in the pre-filled tube 100 of the disclosure configured to draw from about 14 mL to about 40 mL of the blood sample 200, along with the size and materials of the sealing cap 130 and tube 102. The first drug layer 110 may include about 100 mg/mL calcium gluconate and/or calcium chloride as a coagulating agent. The second gel layer may include about 500 mg/mL ascorbic acid as a stabilizing agent. The first drug layer 110 and the second drug layer 112 may each have a volume of about 2.2 mL. As such, first drug layer 110 may include about 220 mg of calcium gluconate and/or calcium chloride, and second drug layer 112 may include about 1220 mg of ascorbic acid. The first gel layer 120 and the second gel layer 122 may each have a volume of about 4.3 mL, or, equivalently, a mass of about 4.5 g. The third drug layer 114 may include ACD-A as an anticoagulant and may have a volume of about 3.6 mL. ACD-A may include about 7 mg/mL citric acid, about 22 mg/mL sodium citrate, and about 24.5 mg/mL dextrose. With this, the third drug layer 114 may include about 25 mg of citric acid, about 79 mg of sodium citrate, and about 88 mg of dextrose. The volume of the tube 102 may be about 54 mL. The tube 102 may be a clear, silicone-coated PRP tube with a size of 29 mm×115 mm. The sealing cap 130 may be a butyl rubber tube stopper that has a size (e.g., diameter) of 29 mm. The pressure of the tube 102 may be about negative 95 kPa. The volume of the space 140 may be about 34 mL. The sealing cap 130 may displace about 3.5 mL of the volume of the tube 102. The ratio of the volume of the blood sample 200 to the combined volume of first drug layer 110, second drug layer 112, and the third drug layer 114 is about 4:1.

### Example 5

TABLE 5
Contents of the PRFM preparation tube 100 configured to draw
from about 2 mL to about 12 mL of the blood sample 200.

| | | | Volume and/or |
|---|---|---|---|
| Item | Description | Size | Mass |
| Tube 102 | PRP tube, clear, | 16 x 125 mm | 16 mL |
| | silicone-coated | | |
| First drug | Calcium | — | 0.7 mL |
| layer 110 | gluconate and/or | | |
| | calcium | | |
| | chloride, USP 10%, | | |
| | 100 mg/mL | | |
| Gel layer | Thixotropic | — | 1.5 g (about 1.4 |
| 120 | separation serum | | mL) |
| | gel, non-sterile | | |
| Second drug | Ascorbic acid | — | 0.7 mL |
| layer 112 | USP, 500 | | |
| | mg/mL | | |
| Sealing cap | Tube stopper, | 16 mm | — |
| 130 | butyl rubber | | |

The tube 102 of the PRFM preparation tube 100′ may be configured to draw from about 2 mL to about 12 mL of blood (e.g., about 2 mL to about 10 mL, about 2 mL to about 8 mL, about 2 mL to about 4 mL, about 4 mL to about 8 mL, about 4 mL to about 12 mL, about 8 mL to about 10 mL, about 10 mL to about 12 mL, e.g., about 2 mL, about 3 mL, about 4 mL, about 5 mL, about 6 mL, about 7 mL, about 8 mL, about 9 mL, about 10 mL, about 11 mL, or about 12 mL of blood) from a subject.

Table 5 describes the content of each of the three layers in the pre-filled tube 100′ of the disclosure configured to draw from about 2 mL to about 12 mL of blood, along with the size and materials of sealing cap 130 and tube 102. The first drug layer 110 and the second drug layer 112 can each have a volume of about 0.7 mL. The first drug layer 110 can include calcium gluconate as a coagulating agent and/or calcium chloride at a concentration of about 100 mg/mL. The second drug layer 112 can include ascorbic acid as a coagulating agent at a concentration of about 500 mg/mL. As such, first drug layer 110 can include about 70 mg of calcium gluconate and/or calcium chloride, and second drug layer 112 can include about 350 mg of ascorbic acid.

The gel layer 120 can include a non-sterile, thixotropic separation serum gel and can have a volume of about 1.4 mL, or, equivalently, a mass of about 1.5 g. The volume of tube 102 can be about 16 mL. Tube 102 can be a clear, silicone-coated PRP tube with a size of 16 mm by 125 mm. Sealing cap 130 can be a butyl rubber tube stopper that has a size (e.g., diameter) of 16 mm. The pressure of tube 102 can be about negative 95 kPa. The volume of space 108 may be about 12 mL. The sealing cap 130 may displace about 1 mL of the volume of tube 102. The ratio of the volume of the blood sample 200 to the combined volume of first drug layer 110 and second drug layer 112 is about 8:1.

### Example 6

TABLE 6
Contents of the pre-filled tube 100 configured to draw
from about 7 mL to about 22 mL of the blood sample 200.

| | | | Volume and/or |
|---|---|---|---|
| Item | Description | Size | Mass |
| Tube 102 | PRP tube, clear, | 20 x 125 mm | 32 mL |
| | silicone-coated | | |
| First drug | Calcium | — | 1.3 mL |
| layer 110 | gluconate and/or | | |
| | calcium | | |
| | chloride, USP | | |
| | 10%, 100 | | |
| | mg/mL | | |
| Gel layer | Thixotropic | — | 4 g (about 3.8 |
| 120 | separation serum | | mL) |
| | gel, non-sterile | | |
| Second drug | Ascorbic acid | — | 1.3 mL |
| layer 112 | USP, 500 | | |
| | mg/mL | | |
| Sealing cap | Tube stopper, | 20 mm | — |
| 130 | butyl rubber | | |

The tube 102 of the pre-filled tube 100′ may be configured to draw from about 7 mL to about 22 mL of the blood sample 200 (e.g., from about 7 mL to about 10 mL, about 10 mL to about 18 mL, about 8 mL to about 12 mL, from about 18 mL to about 20 mL, from about 19 mL to about 20 mL, from about 20 mL to about 22 mL, e.g., about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 21 mL, or about 22 mL of the blood sample 200) from a subject. As little as 2.0 mL of the blood sample 200 may be collected (e.g., if the vein collapses during the blood draw).

Table 6 describes the content of each of the three layers in the pre-filled tube 100′ of the disclosure configured to draw from about 7 mL to about 22 mL of the blood sample 200, along with the size and materials of the sealing cap 130 and tube 102. First drug layer 110 and second drug layer 112 may each have a volume of about 1.3 mL. First drug layer 110 may include calcium gluconate and/or calcium chloride as a coagulating agent at a concentration of about 100 mg/mL. The second drug layer 112 may include ascorbic acid as a stabilizing agent at a concentration of about 500 mg/mL. As such, the first drug layer 110 may include about 130 mg of calcium gluconate and/or calcium chloride, and the second drug layer 112 may include about 650 mg of ascorbic acid. The gel layer 120 may include a non-sterile, thixotropic separation serum gel and may have a volume of about 4 mL, or, equivalently, a mass of about 3.8 g. The volume of the tube 102 may be about 32 mL. The tube 102 may be a clear, silicone-coated PRP tube with a size of 20×125 mm. Sealing cap 130 may be a butyl rubber tube stopper that has a size (e.g., diameter) of 20 mm. The pressure of the tube 102 may be about negative 95 kPa. The volume of the space 108 may be about 24 mL. The sealing cap 130 may displace about 1.9 mL of the volume of the tube 102. The ratio of the volume of the blood to the combined volume of first drug layer 110 and second drug layer 112 is about 8:1.

### Example 7

TABLE 7
Contents of the pre-filled tube 100′ configured
to draw from about 12 mL to about 36 mL of blood.

| | | | Volume and/or |
|---|---|---|---|
| Item | Description | Size | Mass |
| Tube 102 | PRP tube, clear, | 29 x 115 mm | 54 mL |
| | silicone-coated | | |
| First drug | Calcium | — | 2 mL |
| layer 110 | gluconate and/or | | |
| | calcium | | |
| | chloride, USP 10%, | | |
| | 100 mg/mL | | |
| Gel layer | Thixotropic | — | 9 g (about 8.6 |
| 120 | separation serum | | mL) |
| | gel, non-sterile | | |
| Second drug | Ascorbic acid | — | 2 mL |
| layer 112 | USP, 500 | | |
| | mg/mL | | |
| Sealing cap | Tube stopper, | 29 mm | — |
| 130 | butyl rubber | | |

The tube 102 of the pre-filled tube 100′ may be configured to draw from about 12 mL to about 36 mL of blood (e.g., from about 12 mL to about 30 mL, from about 12 mL to about 20 mL, from about 15 mL to about 30 mL, from about 15 mL to about 20 mL, from about 30 mL to about 33 mL, from about 33 mL to about 36 mL, from about 32 mL to about 36 mL, from about 34 mL to about 36 mL, from about 30 mL to about 32 mL, or from about 35 mL to about 36 mL, e.g., about 12 mL, about 13 mL, about 14 mL, about 15 mL, about 16 mL, about 17 mL, about 18 mL, about 19 mL, about 20 mL, about 21 mL, about 22 mL, about 23 mL, about 24 mL, about 25 mL, about 26 mL, about 27 mL, about 28 mL, about 29 mL, about 30 mL, about 31 mL, about 32 mL, about 33 mL, about 34 mL, about 35 mL, or about 36 mL of blood) from a subject. As little as 2.0 mL of blood may be collected (e.g., if the vein collapses during the blood draw).

Table 7 describes the content of each of the three layers in the pre-filled tube 100′ of the disclosure configured to draw from about 12 mL to about 36 mL of blood, along with the size and materials of the sealing cap 130 and tube 102. The first drug layer 110 and the second drug layer 112 may each have a volume of about 2 mL. The first drug layer 110 may include calcium gluconate and/or calcium chloride as a coagulating agent at a concentration of about 100 mg/mL. The second drug layer 112 may include ascorbic acid as a stabilizing agent at a concentration of about 500 mg/mL. As such, the first drug layer 110 may include about 200 mg of calcium gluconate and/or calcium chloride, and second drug layer 112 may include about 1000 mg of ascorbic acid. The gel layer 120 may include a non-sterile, thixotropic separation serum gel and may have a volume of about 8.5 mL, or, equivalently, a mass of about 9 g. The volume of the tube 102 may be about 54 mL. The tube 102 may be a clear, silicone-coated PRP tube with a size of 29×115 mm. The sealing cap 130 may be a butyl rubber tube stopper that has a size (e.g., diameter) of 29 mm. The pressure of the tube 102 may be about negative 85 kPa. The volume of the space 108 may be about 38 mL. The sealing cap 130 may displace about 3.5 mL of the volume of the tube 102. The ratio of the volume of the blood to the combined volume of first drug layer 110 and the second drug layer 112 is about 8:1.

### Example 8

TABLE 8
Contents of the pre-filled tube 100′ configured
to draw from about 14 mL to about 40 mL of blood.

| | | | Volume and/or |
|---|---|---|---|
| Item | Description | Size | Mass |
| Tube 102 | PRP tube, clear, | 29 x 115 mm | 54 mL |
| | silicone-coated | | |
| First drug | Calcium | — | 2.2 mL |
| layer 110 | gluconate and/or | | |
| | calcium | | |
| | chloride, USP | | |
| | 10%, 100 | | |
| | mg/mL | | |
| Gel layer | Thixotropic | — | 9 g (about 8.5 |
| 120 | separation serum | | mL) |
| | gel, non-sterile | | |
| Second drug | Ascorbic acid | — | 2.2 mL |
| layer 112 | USP, 500 | | |
| | mg/mL | | |
| Sealing cap | Tube stopper, | 29 m | — |
| 130 | butyl rubber | | |

The tube 102 of the pre-filled tube 100′ may be configured to draw from about 14 mL to about 40 mL of blood (e.g., from about 14 mL to about 20 mL, from about 15 mL to about 30 mL, from about 15 mL to about 25 mL, from about 25 mL to about 30 mL, from about 20 mL to about 30 mL, from about 31 mL to about 35 mL, from about 35 mL to about 38 mL, from about 38 mL to about 40 mL, from about 34 mL to about 35 mL, from about 34 mL to about 36 mL, from about 34 mL to about 38 mL, from about 36 mL to about 40 mL, e.g., about 14 mL, about 15 mL, about 16 mL, about 17 mL, about 18 mL, about 19 mL, about 20 mL, about 21 mL, about 22 mL, about 23 mL, about 24 mL, about 25 mL, about 26 mL, about 27 mL, about 28 mL, about 29 mL, about 30 mL, about 31 mL, about 32 mL, about 33 mL, about 34 mL, about 35 mL, about 36 mL, about 37 mL, about 38 mL, about 39 mL, or about 40 mL of blood) from a subject. As little as 2.0 mL of blood may be collected (e.g., if the vein collapses during the blood draw).

Table 8 describes the content of each of the three layers in the pre-filled tube 100′ of the disclosure configured to draw from about 14 mL to about 40 mL of blood, along with the size and materials of the sealing cap 130 and tube 102. The first drug layer 110 and the second drug layer 112 may each have a volume of about 2.2 mL. The first drug layer 110 may include calcium gluconate and/or calcium chloride as a coagulating agent at a concentration of about 100 mg/mL. The second drug layer 112 may include ascorbic acid as a stabilizing agent at a concentration of about 500 mg/mL. As such, the first drug layer 110 may include 220 mg of calcium gluconate and/or calcium chloride, and the second drug layer 112 may include 1,100 mg of ascorbic acid. The gel layer 120 may include a non-sterile, thixotropic separation serum gel and may have a volume of about 8.5 mL, or, equivalently, a mass of about 9 g. The volume of the tube 102 may be about 54 mL. The tube 102 may be a clear, silicone-coated PRP tube with a size of 29×115 mm. The sealing cap 130 may be a butyl rubber tube stopper that has a size (e.g., diameter) of 29 mm. The pressure of the tube 102 may be about negative 95 kPa. The volume of the space 108 may be about 38 mL. The sealing cap 130 may displace about 3.5 mL of the volume of the tube 102. The ratio of the volume of the blood to the combined volume of first drug layer 110 and second drug layer 112 is about 8:1.

### Example 9

TABLE 9
Contents of the pre-filled tube 100′ configured
to draw from about 2 mL to about 12 mL of blood.

| | | | Volume and/or |
|---|---|---|---|
| Item | Description | Size | Mass |
| Tube 102 | PRP tube, clear, | 16 × 125 mm | 16 mL |
| | silicone-coated | | |
| First drug | Calcium | — | 0.7 mL |
| layer 110 | gluconate and/or | | |
| | calcium | | |
| | chloride, USP 10%, | | |
| | 100 mg/mL | | |
| Gel layer | Thixotropic | — | 1.5 g (about 1.4 |
| 120 | separation serum | | mL) |
| | gel, non-sterile | | |
| Second drug | Anticoagulant | — | 1.1 mL |
| layer 112 | citrate dextrose | | |
| | “A” (ADC-A) | | |
| Sealing cap | Tube stopper, | 16 m | — |
| 130 | butyl rubber | | |

The tube 102 of the pre-filled tube 100′ may be configured to draw from about 2 mL to about 12 mL of blood (e.g., about 2 mL to about 10 mL, about 2 mL to about 8 mL, about 2 mL to about 4 mL, about 4 mL to about 8 mL, about 4 mL to about 12 mL, about 8 mL to about 10 mL, about 10 mL to about 12 mL, e.g., about 2 mL, about 3 mL, about 4 mL, about 5 mL, about 6 mL, about 7 mL, about 8 mL, about 9 mL, about 10 mL, about 11 mL, or about 12 mL of blood) from a subject. As little as 2.0 mL of blood may be collected (e.g., if the vein collapses during the blood draw).

Table 9 describes the content of each of the three layers in the pre-filled tube 100′ of the disclosure configured to draw from about 2 mL to about 12 mL of blood, along with the size and materials of the sealing cap 130 and the tube 102. The first drug layer 110 may include calcium gluconate and/or calcium chloride as a coagulating agent at a concentration of 100 mg/mL and may have a volume of about 0.7 mL. As such, the first drug layer 110 may include about 70 mg of calcium gluconate and/or calcium chloride. The gel layer 120 may include a non-sterile, thixotropic separation serum gel and may have a volume of about 1.4 mL, or, equivalently, a mass of about 1.5 g. The second drug layer 112 may include ACD-A as an anticoagulant and may have a volume of about 1.1 mL. ACD-A may include about 7 mg/mL citric acid, about 22 mg/mL sodium citrate, and about 24.5 mg/mL dextrose. With this, the second drug layer 112 may include about 8 mg of citric acid, about 24 mg of sodium citrate, and about 27 mg of dextrose. The volume of the tube 102 may be about 16 mL. The tube 102 may be a clear, silicone-coated PRP tube with a size of 16×125 mm. Sealing cap 130 may be a butyl rubber tube stopper that has a size (e.g., diameter) of 16 mm. The pressure of the tube 102 may be about negative 95 kPa. The volume of the space 108 may be about 12 mL. Sealing cap 130 may displace about 1 mL of the volume of the tube 102. The ratio of the volume of the blood to the combined volume of the first drug layer 110 and the second drug layer 112 is about 6:1.

### Example 10

TABLE 10
Contents of the pre-filled tube 100′ configured
to draw from about 7 mL to about 22 mL of blood.

| | | | Volume and/or |
|---|---|---|---|
| Item | Description | Size | Mass |
| Tube 102 | PRP tube, clear, | 20 x 125 mm | 32 mL |
| | silicone-coated | | |
| First drug | Calcium | — | 1.3 mL |
| layer 110 | gluconate and/or | | |
| | calcium | | |
| | chloride, USP 10%, | | |
| | 100 mg/mL | | |
| | mg/mL | | |
| Gel layer | Thixotropic | — | 4 g (about 3.8 |
| 120 | separation serum | | mL) |
| | gel, non-sterile | | |
| Second drug | Anticoagulant | — | 2.2 mL |
| layer 112 | citrate dextrose | | |
| | “A” (ADC-A) | | |
| Sealing cap | Tube stopper, | 20 m | — |
| 130 | butyl rubber | | |

The tube 102 of the pre-filled tube 100′ may be configured to draw from about 7 mL to about 22 mL of blood (e.g., from about 7 mL to about 10 mL, about 10 mL to about 18 mL, about 8 mL to about 12 mL, from about 18 mL to about 20 mL, from about 19 mL to about 20 mL, from about 20 mL to about 22 mL, e.g., about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 21 mL, or about 22 mL of blood) from a subject. As little as 2.0 mL of blood may be collected (e.g., if the vein collapses during the blood draw).

Table 10 describes the content of each of the three layers in the pre-filled tube 100′ of the disclosure configured to draw from about 7 mL to about 22 mL of blood, along with the size and materials of the sealing cap 130 and tube 102. The first drug layer 110 may include calcium gluconate and/or calcium chloride as a coagulating agent at a concentration of 100 mg/mL and may have a volume of about 1.3 mL. As such, the first drug layer 110 may include about 130 mg of calcium gluconate and/or calcium chloride. The gel layer 120 may include a non-sterile, thixotropic separation serum gel and may have a volume of about 3.8 mL, or, equivalently, a mass of about 4 g. The second drug layer 112 may include ACD-A as an anticoagulant and may have a volume of about 2.2 mL. ACD-A may include about 7 mg/mL citric acid, about 22 mg/mL sodium citrate, and about 24.5 mg/mL dextrose. With this, the second drug layer 112 may include about 15 mg of citric acid, about 48 mg of sodium citrate, and about 54 mg of dextrose. The volume of the tube 102 may be about 32 mL. The tube 102 may be a clear, silicone-coated PRP tube with a size of 20×125 mm. The sealing cap 130 may be a butyl rubber tube stopper that has a size (e.g., diameter) of 20 mm. The pressure of the tube 102 may be about negative 95 kPa. The volume of the space 108 may be about 23 mL. The sealing cap 130 may displace about 1.9 mL of the volume of the tube 102. The ratio of the volume of the blood to the combined volume of drug layer 110 and the second drug layer 112 is about 6:1.

### Example 11

TABLE 11
Contents of the pre-filled tube 100′ configured
to draw from about 12 mL to about 36 mL of blood.

| | | | Volume and/or |
|---|---|---|---|
| Item | Description | Size | Mass |
| Tube 102 | PRP tube, clear, | 29 x 115 mm | 54 mL |
| | silicone-coated | | |
| First drug | Calcium | — | 2 mL |
| layer 110 | gluconate and/or | | |
| | calcium | | |
| | chloride, USP 10%, | | |
| | 100 mg/mL | | |
| Gel layer | Thixotropic | — | 9 g (about 8.6 |
| 120 | separation serum | | mL) |
| | gel, non-sterile | | |
| Second drug | Anticoagulant | — | 3.2 mL |
| layer 112 | citrate dextrose | | |
| | “A” (ADC-A) | | |
| Sealing cap | Tube stopper, | 29 mm | — |
| 130 | butyl rubber | | |

The tube 102 of the pre-filled tube 100′ may be configured to draw from about 12 mL to about 36 mL of blood (e.g., from about 12 mL to about 30 mL, from about 12 mL to about 20 mL, from about 15 mL to about 30 mL, from about 15 mL to about 20 mL, from about 30 mL to about 33 mL, from about 33 mL to about 36 mL, from about 32 mL to about 36 mL, from about 34 mL to about 36 mL, from about 30 mL to about 32 mL, or from about 35 mL to about 36 mL, e.g., about 12 mL, about 13 mL, about 14 mL, about 15 mL, about 16 mL, about 17 mL, about 18 mL, about 19 mL, about 20 mL, about 21 mL, about 22 mL, about 23 mL, about 24 mL, about 25 mL, about 26 mL, about 27 mL, about 28 mL, about 29 mL, about 30 mL, about 31 mL, about 32 mL, about 33 mL, about 34 mL, about 35 mL, or about 36 mL of blood) from a subject. As little as 2.0 mL of blood may be collected (e.g., if the vein collapses during the blood draw).

Table 11 describes the content of each of the three layers in the pre-filled tube 100′ of the disclosure configured to draw from about 12 mL to about 36 mL of blood, along with the size and materials of the sealing cap 130 and tube 102. The first drug layer 110 may include calcium gluconate and/or calcium chloride as a coagulating agent at a concentration of about 100 mg/mL and may have a volume of about 2 mL. As such, the first drug layer 110 may include about 200 mg of calcium gluconate and/or calcium chloride. The gel layer 120 may include a non-sterile, thixotropic separation serum gel and may have a volume of about 8.6 mL, or, equivalently, a mass of about 9 g. The second drug layer 112 may include ACD-A as an anticoagulant and may have a volume of about 3.2 mL. ACD-A may include about 7 mg/mL citric acid, about 22 mg/mL sodium citrate, and about 24.5 mg/mL dextrose. With this, the second drug layer 112 may include about 22 mg of citric acid, about 70 mg of sodium citrate, and about 78 mg of dextrose. The volume of the tube 102 may be about 54 mL. The tube 102 may be a clear, silicone-coated PRP tube with a size of 29×115 mm. The sealing cap 130 may be a butyl rubber tube stopper that has a size (e.g., diameter) of 29 mm. The pressure of the tube 102 may be about negative 85 kPa. The volume of the space 108 may be about 37 mL. The sealing cap 130 may displace about 3.5 mL of the volume of the tube 102. The ratio of the volume of the blood to the combined volume of drug layer 110 and the second drug layer 112 is about 6:1.

### Example 12

TABLE 12
Contents of the pre-filled tube 100′ configured
to draw from about 14 mL to about 40 mL of blood.

| | | | Volume and/or |
|---|---|---|---|
| Item | Description | Size | Mass |
| Tube 102 | PRP tube, clear, | 29 x 115 mm | 54 mL |
| | silicone-coated | | |
| First drug | Calcium | — | 2.2 mL |
| layer 110 | gluconate and/or | | |
| | calcium | | |
| | chloride, USP 10%, | | |
| | 100 mg/mL | | |
| Gel layer | Thixotropic | — | 9 g (about 8.6 |
| 120 | separation serum | | mL) |
| | gel, non-sterile | | |
| Second drug | Anticoagulant | — | 3.6 mL |
| layer 112 | citrate dextrose | | |
| | “A” (ADC-A) | | |
| Sealing cap | Tube stopper, | 29 mm | — |
| 130 | butyl rubber | | |

The tube 102 of the pre-filled tube 100′ may be configured to draw from about 14 mL to about 40 mL of blood (e.g., from about 14 mL to about 20 mL, from about 15 mL to about 30 mL, from about 15 mL to about 25 mL, from about 25 mL to about 30 mL, from about 20 mL to about 30 mL, from about 31 mL to about 35 mL, from about 35 mL to about 38 mL, from about 38 mL to about 40 mL, from about 34 mL to about 35 mL, from about 34 mL to about 36 mL, from about 34 mL to about 38 mL, from about 36 mL to about 40 mL, e.g., about 14 mL, about 15 mL, about 16 mL, about 17 mL, about 18 mL, about 19 mL, about 20 mL, about 21 mL, about 22 mL, about 23 mL, about 24 mL, about 25 mL, about 26 mL, about 27 mL, about 28 mL, about 29 mL, about 30 mL, about 31 mL, about 32 mL, about 33 mL, about 34 mL, about 35 mL, about 36 mL, about 37 mL, about 38 mL, about 39 mL, or about 40 mL of the blood sample 200) from a subject. As little as 2.0 mL of the blood sample 200 may be collected (e.g., if the vein collapses during the blood draw).

Table 12 describes the content of each of the three layers in the pre-filled tube 100′ of the disclosure configured to draw from about 14 mL to about 40 mL of the blood sample 200, along with the size and materials of the sealing cap 130 and tube 102. The first drug layer 110 may include calcium gluconate and/or calcium chloride as a coagulating agent at a concentration of about 100 mg/mL and may have a volume of about 2.2 mL. As such, the first drug layer 110 may include about 220 mg of calcium gluconate and/or calcium chloride. The gel layer 120 may include a non-sterile, thixotropic separation serum gel and may have a volume of about 8.5 mL, or, equivalently, a mass of about 9 g. The third drug layer 112 may include ACD-A as an anticoagulant and may have a volume of about 3.6 mL. ACD-A may include about 7 mg/mL citric acid, about 22 mg/mL sodium citrate, and about 24.5 mg/mL dextrose. With this, the third drug layer 114 may include about 25 mg of citric acid, about 79 mg of sodium citrate, and about 88 mg of dextrose. The volume of the tube 102 may be about 54 mL. Tube 102 may be a clear, silicone-coated PRP tube with a size of 29×115 mm. The sealing cap 130 may be a butyl rubber tube stopper that has a size (e.g., diameter) of 29 mm. The pressure of the tube 102 may be about negative 95 kPa. The volume of the space 108 may be about 37 mL. The sealing cap 130 may displace about 3.5 mL of the volume of the tube 102. The ratio of the volume of the blood to the combined volume of the first drug layer 110 and the second drug layer 112 is about 6:1.

### OTHER EMBODIMENTS

Other embodiments are in the claims. All publications, patents, and patent applications mentioned in the above specification are hereby incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Other embodiments are within the claims.

1. A method comprising:
introducing a blood sample into a tube, wherein the blood sample, when in the tube, is separated from a stabilizing agent and a coagulating agent with at least one gel layer;
centrifuging the tube to pass red blood cells of the blood sample through the at least one gel layer and to form a mixture of the stabilizing agent, the coagulating agent, and plasma of the blood sample positioned above the at least one gel layer; and
resuspending platelets of the blood sample into the mixture to form a platelet rich fibrin matrix (PRFM) layer in the tube.

2. The method of claim 1, further comprising:
removing at least a portion of the PRFM layer from the tube in a fluid state with a syringe; and
applying the at least the portion of the PRFM layer to a wound site.

3. The method of claim 2, wherein the PRFM layer is removed from the tube having a viscosity less than about 100 cP.

4. The method of claim 1, further comprising:
removing at least a portion of the PRFM layer from the tube in a gel state; and
applying the at least the portion of the PRFM layer to a wound site.

5. The method of claim 4, wherein the PRFM layer is removed from the tube having a viscosity greater than about 100 cP.

6. The method of claim 1, wherein the coagulating agent activates the platelets to form the PRFM layer, and the stabilizing agent stabilizes the PRFM layer.

7. The method of claim 1, wherein the at least one gel layer includes a first gel layer separating the coagulating agent from the stabilizing agent and a second gel layer separating the coagulating agent and the stabilizing agent from the blood sample.

8. The method of claim 1, further comprising mixing the blood sample with an anticoagulant in the tube before centrifuging.

9. The method of claim 8, wherein the anticoagulant comprises anticoagulant citrate dextrose A (ACD-A), buffered citrate, sodium citrate, citrate phosphate dextrose (CPD), ethylenediaminetetraacetic acid (EDTA), heparin, oxolate, a derivative thereof, or any combination thereof.

10. The method of claim 9, wherein the anticoagulant comprises ACD-A.

11. The method of claim 1, wherein the stabilizing agent comprises ascorbic acid.

12. The method of claim 1, wherein the coagulating agent comprises a calcium salt.

13. The method of claim 12, wherein the calcium salt comprises calcium gluconate and/or calcium chloride.

14. The method of claim 1, wherein the tube is prefilled with the stabilizing agent, the coagulating agent, and the at least one gel layer.

15. The method of claim 14, wherein the tube is prefilled with an anti-coagulant agent.

16. The method of claim 14, wherein the coagulating agent is prefilled in the tube at a concentration of from about 50 mg/mL to about 150 mg/mL, inclusive.

17. The method of claim 1, wherein the centrifuging is the only centrifugation.

18. A pre-filled tube configured to prepare a platelet rich fibrin matrix (PRFM) layer, wherein the pre-filled tube comprises:
a first drug layer comprising a coagulating agent;
a second drug layer comprising a stabilizing agent; and
a gel layer separating the first drug layer and the second drug layer.

19. The pre-filled tube of claim 18, wherein the pre-filled tube further comprises:
a third drug layer comprising an anticoagulant; and
a second gel layer separating the third drug layer from the first drug layer and the second drug layer.

20. The pre-filled tube of claim 19, wherein the anticoagulant comprises anticoagulant citrate dextrose A (ACD-A), buffered citrate, sodium citrate, citrate phosphate dextrose (CPD), ethylenediaminetetraacetic acid (EDTA), heparin, oxolate, a derivative thereof, or any combination thereof.

21. The pre-filled tube of claim 20, wherein the anticoagulant comprises ACD-A.

22. The pre-filled tube of claim 18, wherein the coagulating agent comprises a calcium salt.

23. The pre-filled tube of claim 22, wherein the calcium salt is calcium gluconate and/or calcium chloride.

24. The pre-filled tube of claim 18, wherein the coagulating agent has a concentration of from about 50 mg/mL to about 150 mg/mL, inclusive.

25. The pre-filled tube of claim 18, wherein the stabilizing agent is ascorbic acid.

26. The pre-filled tube of claim 18, wherein the first drug layer is closer to a distal end of the pre-filled tube than the second drug layer.

27. The pre-filled tube of claim 18, wherein the second drug layer is closer to a distal end of the pre-filled tube than the first drug layer.

28. The pre-filled tube of claim 18, wherein the pre-filled tube is sealed at a proximal end with a sealing cap to create a vacuum inside of the pre-filled tube, and the pre-filled tube contains an air pressure from about negative 50 kPa to about negative 101 kPa, inclusive.
